SEQUENCE LISTING (2 sequences)

SEQ ID NO: 1 - RNA, 21 nt, synthetic construct
  misc_feature 1..21: note = Synthetic
  modified_base 1: mod_base = OTHER; note = 2'-fluoroguanosine, 3'-5' phosphorothioate linkage
  modified_base 2: mod_base = OTHER; note = 2'-O-methylguanosine, 3'-5' phosphorothioate linkage
  modified_base 3: mod_base = OTHER; note = 2'-fluorouridine
  modified_base 4: mod_base = OTHER; note = 2'-O-methyluridine
  modified_base 5: mod_base = OTHER; note = 2'-fluoroadenosine
  modified_base 6: mod_base = OTHER; note = 2'-O-methyladenosine
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluoroadenosine
  modified_base 12: mod_base = OTHER; note = 2'-O-methyluridine
  modified_base 13: mod_base = OTHER; note = 2'-fluorouridine
  modified_base 14: mod_base = OTHER; note = 2'-O-methyluridine
  modified_base 15: mod_base = OTHER; note = 2'-fluoroadenosine
  modified_base 16: mod_base = OTHER; note = 2'-O-methylcytidine
  modified_base 17: mod_base = OTHER; note = 2'-fluorouridine
  modified_base 18: mod_base = OTHER; note = 2'-O-methyluridine
  modified_base 19: mod_base = OTHER; note = 2'-fluorocytidine
  modified_base 20: mod_base = OTHER; note = 2'-O-methyladenosine
  modified_base 21: mod_base = OTHER; note = 2'-fluoroadenosine, L96
  modified_base 1..21: mod_base = OTHER; note = 3'-5' phosphodiester linkage
  source 1..21: mol_type = other RNA
ggttaacaccatttacttcaa

SEQ ID NO: 2 - RNA, 23 nt, synthetic construct
  misc_feature 1..23: note = Synthetic
  modified_base 1: mod_base = OTHER; note = 2'-O-methyluridine, 3'-5' phosphorothioate linkage
  modified_base 2: mod_base = OTHER; note = 2'-fluorouridine, 3'-5' phosphorothioate linkage
  modified_base 3: mod_base = OTHER; note = 2'-O-methylguanosine
  modified_base 4: mod_base = OTHER; note = 2'-fluoroadenosine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyluridine
  modified_base 8: mod_base = OTHER; note = 2'-fluoroadenosine
  modified_base 9: mod_base = OTHER; note = 2'-O-methyladenosine
  modified_base 10: mod_base = OTHER; note = 2'-fluoroadenosine
  modified_base 11: mod_base = OTHER; note = 2'-O-methyluridine
  modified_base 12: mod_base = OTHER; note = 2'-O-methylguanosine
  modified_base 13: mod_base = OTHER; note = 2'-O-methylguanosine
  modified_base 14: mod_base = OTHER; note = 2'-fluorouridine
  modified_base 15: mod_base = OTHER; note = 2'-O-methylguanosine
  modified_base 16: mod_base = OTHER; note = 2'-fluorouridine
  modified_base 17: mod_base = OTHER; note = 2'-O-methyluridine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorocytidine
  modified_base 21: mod_base = OTHER; note = 2'-O-methylcytidine, 3'-5' phosphorothioate linkage
  modified_base 22: mod_base = OTHER; note = 2'-O-methyladenosine, 3'-5' phosphorothioate linkage
  modified_base 23: mod_base = OTHER; note = 2'-O-methylguanosine
  modified_base 1..23: mod_base = OTHER; note = 3'-5' phosphodiester linkage
  source 1..23: mol_type = other RNA
ttgaagtaaatggtgttaaccag

(19) United States
(12) Patent Application Publication
(10) Pub. No.: US 2025/0064842 A1
(43) Pub. Date: Feb. 27, 2025

(54) Fitusiran for Use in Improving Patient-Reported Outcome in Hemophilia Patients

(71) Applicant: Genzyme Corporation, Cambridge, MA (US)

(72) Inventors: Shauna Andersson, Lexington, MA (US); Jose Bartelt-Hofer, Lyon, Rhone (FR); Pronabesh Dasmahapatra, Somerville, MA (US)

(73) Assignee: Genzyme Corporation, Cambridge, MA (US)

(21) Appl. No.: 18/723,352

(22) PCT Filed: Dec. 22, 2022
(86) PCT No.: PCT/US2022/082213

§ 371 Date: Jun. 21, 2024

Related U.S. Application Data
(60) Provisional application No. 63/386,489, filed on Dec. 7, 2022.
(60) Provisional application No. 63/382,223, filed on Nov. 3, 2022.
(60) Provisional application No. 63/359,717, filed on Jul. 8, 2022.
(60) Provisional application No. 63/335,535, filed on Apr. 27, 2022.
(60) Provisional application No. 63/305,550, filed on Feb. 1, 2022.
(60) Provisional application No. 63/293,070, filed on Dec. 22, 2021.

(51) Int. Cl. A61K 31/7056; A61P 7/04

(52) U.S. Cl. CPC A61K 31/7056; A61P 7/04

(57) ABSTRACT

The present disclosure provides using a double-stranded oligonucleotide compound as a novel therapy to improve the patient reported outcomes and quality of life of patients with hemophilia A or hemophilia B.

### CROSS-REFERENCE TO RELATED APPLICATIONS

The present application claims priority from U.S. Applications 63/293,070, filed Dec. 22, 2021; 63/305,550, filed Feb. 1, 2022; 63/335,535, filed Apr. 27, 2022; 63/359,717, filed Jul. 8, 2022; 63/382,223, filed Nov. 3, 2022; and 63/386,489, filed Dec. 7, 2022. The contents of the priority applications are incorporated by reference herein in their entirety.

### SEQUENCE LISTING

The instant application contains a Sequence Listing which has been submitted electronically in XML format and is hereby incorporated by reference in its entirety. Said XML copy, created on Dec. 21, 2022, is named 022548_WO087_SL.xml and is 28,905 bytes in size.

### BACKGROUND OF THE INVENTION

Maintenance of normal hemostasis relies on a regulated set of simultaneously occurring procoagulant and anticoagulant processes, in which thrombin plays a central role. Hemophilia A and B are inherited bleeding disorders characterized by the body's inability to control blood clotting. They are caused by deficiencies in factors VIII and IX, respectively. Bleeding in hemophilia A and B arises from insufficient thrombin generation (Peyvandi et al., Lancet (2016) 388(10040):187-97). Without effective treatment, patients with hemophilia experience recurrent bleeding, which lead to major disability due to chronic hemarthropathy and significant pain, and can be life-threatening (Pipe et al., Haemophilia (2007) 13 Suppl 4:1-16).

Significant unmet needs and management challenges continue to exist for all hemophilia populations despite treatment advances. While prophylaxis based on replacement therapy with factor VIII or IX is considered the cornerstone of hemophilia management, it has significant limitations. For example, injections of factor replacement for prophylaxis are burdensome and impractical, often requiring multiple intravenous infusions per week (Peyvandi, supra; Ljung and Andersson, Br J Haematol. (2015) 169(6):777-86; Srivastava et al., Haemophilia (2013) 19(1):e1-47; Bauer, Am J Manag Care (2015) 21(6 Suppl):S112-22; Mannucci and Franchini, Blood Transfus. (2013) 11(Suppl 4):s77-81). Factor replacement is also limited by difficulties with venous access and risk of infections (Balkaransingh and Young, Ther Adv Hematol. (2018) 9(2):49-61; Valentino et al., Blood Rev. (2011) 25(1):11-5). Limitations in delivering factor replacement also result in a large proportion of the world's hemophilia population without access in the first instance to prophylaxis treatment (Hemophilia, W.F.O. Treatment Safety and Supply. 2020).

Additionally, treatment with factor replacement products can result in the development of inhibitory alloantibodies, rendering the factor treatment ineffective (Morfini et al., Haemophilia (2007) 13(5):606-12). These inhibitors, which typically occur in childhood, limit treatment options and dramatically worsen the prognosis of hemophilia. Moreover, those with persistent inhibitors typically have a lower quality of life, greater joint disease, greater surgical risk, and higher mortality, including a higher risk of death from hemophilia-related bleeding complications, when compared to patients without inhibitors (Morfini, supra; Oladapo et al., Orphanet J Rare Dis. (2018) 13(1):198). Current treatment strategies for individuals with persistent inhibitors include immune tolerance induction (ITI) and prophylaxis with bypassing agents (BPAs) such as activated prothrombin complex concentrates (aPCC) and recombinant activated factor VII (rFVIIa) (Benson et al., Eur J Haematol. (2012) 88(5):371-79; Collins et al., Br J Haematol. (2013) 160(2):153-70; Kempton et al., Blood (2014) 124(23):3365-72; Astermark et al., Haemophilia (2007) 13(1):38-45; Eichinger et al., Eur J Clin Invest. (2009) 39(8):707-13).

There remains an urgent and unmet need to develop therapeutics and treatment methods that prevent recurrent bleeding and improve the overall quality of life for patients with hemophilia.

### SUMMARY OF THE INVENTION

The present disclosure provides methods and compositions for treating hemophilia patients. In one aspect, the present disclosure provides a method of improving patient-reported outcome (PRO) in a hemophilia patient (e.g., an adult or adolescent patient twelve years or older), comprising subcutaneously administering a therapeutically effective amount of fitusiran to a hemophilia patient in need thereof, optionally wherein the patient is a hemophilia A or B patient with or without inhibitors and optionally wherein the PRO is improved in one or more quality of life (QoL) domains. In a further aspect, the present disclosure provides a method of improving quality of life (QoL) in a hemophilia patient (e.g., an adult or adolescent patient twelve years or older), comprising subcutaneously administering fitusiran to a hemophilia patient in need thereof, wherein the QoL is improved in one or more QoL domains and optionally wherein the patient is a hemophilia A or B patient with or without inhibitors. In some embodiments, fitusiran is administered at about 10 to about 90 mg per dose.

In some embodiments, the one or more QoL domains are domains in a QoL questionnaire. In further embodiments, the QoL questionnaire is Haemophilia Quality of Life Questionnaire for Adults (Haem-A-QoL). In certain embodiments, the administering results in a clinically meaningful improvement indicated by a reduction of 7 or more units (optionally 8 or more, 9 or more, or 10 or more units) in one or more of Total Score, Sports and Leisure domain score, and Physical Health domain score of Haem-A-QoL.

In some embodiments, the QoL questionnaire is Haemophilia Quality of Life Questionnaire for children and adolescents (Haemo-QoL). In certain embodiments, the administering results in a clinically meaningful improvement indicated by a reduction of 7 or more units (optionally 8 or more, 9 or more, or 10 or more units) in the Total Score of Haemo-QoL.

In some embodiments, the QoL questionnaire is Hemophilia Activities List (HAL). In certain embodiments, the administering results in an improvement indicated by an increase of 7 or more units (optionally 8 or more, 9 or more, or 10 or more units) in the Total Score of HAL.

In some embodiments, the QoL questionnaire is pediatric Hemophilia Activities List (pedHAL). In certain embodiments, the administering results in an improvement indicated by an increase of 7 or more units (optionally 8 or more, 9 or more, or 10 or more units) in the Total Score of pedHAL.

In some embodiments, the QoL questionnaire is Treatment Satisfaction Questionnaire for Medication Version 9 (TSQM-9). In certain embodiments, the administering results in an improvement indicated by an increase of 7 or more units (optionally 8 or more, 9 or more, or 10 or more units) in one or more of Effectiveness, Satisfaction, and Convenience domain score of TSQM-9.

In some embodiments, the QoL questionnaire is EuroQol 5-Dimensions (EQ-5D-5L). In certain embodiments, the administering results in an improvement indicated by an increase of 0.02 or more units (optionally 0.03 or more, 0.04 or more, or 0.05 or more units) in the total score of EQ-5D-5L or an increase of 5 or more units (optionally 6 or more, 7 or more, or 8 or more units) in the visual analog scale (VAS) score of EQ-5D-5L.

In some embodiments, the patient has hemophilia A; in further embodiments, the hemophilia A patient has been treated with factor VIII or a bypassing agent (BPA). In some embodiments, the patient has hemophilia B; in further embodiments, the hemophilia B patient has been treated with factor IX or a BPA.

In some embodiments, the patient is with inhibitors, optionally wherein the level of inhibitors is more than 0.6 BU/mL as determined by Bethesda inhibitor assay. In certain embodiments, the BPA is activated prothrombin complex concentrates (aPCC) and/or recombinant activated Factor VII (rFVIIa). In other embodiments, the patient is without inhibitors.

In some embodiments, the method comprises administering to the patient a plurality of doses of fitusiran at about 10 mg, about 20 mg, about 50 mg, or about 80 mg per dose. In some embodiments, fitusiran is administered to the patient about once every four weeks or about once a month or about once every eight weeks or about once every two months. In some embodiments, fitusiran is provided at a concentration of about 1 to about 200 mg/mL, optionally at a concentration of about 6.25, 12.5, or 100 mg/mL, in a phosphate-buffered saline (pH 7).

In another aspect, the present disclosure provides fitusiran for use in the treatment method herein. In yet another aspect, the present disclosure provides an article of manufacture for use in the treatment methods herein. In some embodiments, the article of manufacture is a single-use container containing about 80 mg of fitusiran in about 0.8 mL of a phosphate-buffered saline (pH 7), about 50 mg of fitusiran in about 0.5 mL of a phosphate-buffered saline (pH 7), about 20 mg of fitusiran in about 0.2 mL of a phosphate-buffered saline (pH 7), or about 10 mg of fitusiran in about 0.1 mL of a phosphate-buffered saline (pH 7). In certain embodiments, the article of manufacture is a single-use prefilled syringe containing about 80 mg of fitusiran in about 0.8 mL of a phosphate-buffered saline (pH 7), about 50 mg of fitusiran in about 0.5 mL of a phosphate-buffered saline (pH 7), about 20 mg of fitusiran in about 0.2 mL of a phosphate-buffered saline (pH 7), or about 10 mg of fitusiran in about 0.1 mL of a phosphate-buffered saline (pH 7). In yet another aspect, the present disclosure provides the use of fitusiran for the manufacture of a medicament to treat hemophilia A or B in the treatment method herein.

### BRIEF DESCRIPTION OF THE DRAWINGS

FIG. 1 shows the expanded structural formula, chemical formula, and molecular mass of fitusiran (sodium form).

FIG. 2 is a diagram showing the design for the study of patients with inhibitors. Abbreviations are as follows: BPA, bypassing agent; HA, hemophilia type A; HB, hemophilia type B; R, randomized. In the on-demand arm, patients are treated with on-demand factor concentrates as routinely prescribed by physician per local standard practice.

FIG. 3 is a diagram showing the design for the study of patients without inhibitors. Abbreviations are as described in FIG. 2.

FIG. 4 is a diagram showing the participant disposition of patients enrolled in the Phase 3 clinical trial of fitusiran in Hemophilia A and B patients with inhibitors.

FIG. 5 is a bar graph showing the important aspects of hemophilia treatment as determined according to patient exit interviews.

FIG. 6 is a bar graph showing improvements with fitusiran prophylaxis treatment according to patient exit interviews.

FIG. 7 is a line graph showing the mean antithrombin levels in patients with inhibitors treated with fitusiran.

FIG. 8 is a line graph showing thrombin generation in patients with inhibitors treated with fitusiran.

FIG. 9 is a line graph showing the mean antithrombin levels in patients without inhibitors treated with fitusiran.

FIG. 10 is a line graph showing thrombin generation in patients without inhibitors treated with fitusiran.

FIG. 11 shows the mean change in peak thrombin generation in patients with (ATLAS-INH) and without (ATLAS-A/B) inhibitors treated with fitusiran.

FIG. 12 shows the median observed ABR for patients treated with fitusiran.

FIG. 13 is a bar graph showing the percentage of patients with zero, or three or less, bleeds during the fitusiran efficacy period.

FIG. 14 shows the mean consumption of factor and BPA by patients during the fitusiran efficacy period.

FIG. 15 shows the percentage of patients with no factor of BPA use during the fitusiran efficacy period.

FIG. 16 shows the total number of treated bleeds and BPA/factor injections in the fitusiran efficacy period.

FIG. 17 shows the mean annualized injection rates of BPAs and factor in patients.

FIG. 18 shows the weight-adjusted doses of BPA and replacement factor administered per bleed during the fitusiran efficacy period.

FIG. 19 shows bar charts showing bleeding events in the efficacy period. Panel A shows annualized bleeding rates for treated bleeds estimated by negative binomial model, and panel B shows median annualized bleeding rates for treated bleeds. Abbreviations are as follows: ABR, annualized bleeding rate; BPA, bypassing agent; CI, confidence interval; IQR, interquartile range.

FIG. 20 is a Forest plot of estimated annualized bleeding rate ratio for treated bleeds in the efficacy period based on on-treatment strategy using negative binomial model by baseline factor (ITT Set). For the hemophilia B subgroup, negative binomial model convergence is questionable due to limited sample size. The efficacy period is from day 29 to day 246, or the last day of bleeding follow up, whichever is the earliest. The analysis is based on on-treatment strategy which excluded any bleeding events in the period of intercurrent events. Abbreviations are as follows: BPA, bypassing agent; ITT, intent-to-treat; NA, not available; RR, rate ratio.

FIG. 21 is a bar graph showing improvement from baseline to end of trial in Haem-A-QoL total and physical health domain transformed scores (secondary endpoint). Clinically meaningful improvements were defined as a seven-point decrease for total score and 10-point decrease for the physical health domain score. Analysis of covariance model includes treatment group and randomization strata of number of bleeds in the six months prior to trial (≤10, >10) as fixed effects, baseline score as a covariate. Haem-A-QoL scores range 0-100; lower scores=improved HRQoL. Abbreviations are as follows: BPA, bypassing agent; CI, confidence interval; Haem-A-QoL, Haemophilia Quality of Life Questionnaire for Adults; HRQoL, health-related quality of life; LS, least-square mean)

FIG. 22 shows mean (±SE) change in antithrombin levels compared to baseline (panel A) and peak thrombin generation compared to baseline (panel B).

### DETAILED DESCRIPTION OF THE INVENTION

The present disclosure features methods of using fitusiran for routine prophylaxis to prevent or reduce the frequency of bleeding episodes in patients with hemophilia, such as hemophilia A (congenital factor VIII deficiency) or hemophilia B (congenital factor IX deficiency), with or without inhibitors. In some embodiments, the patient is an adult or adolescent ≥12 years old. These methods increase the quality of life of hemophilia patients. In particular embodiments, the present methods improve the patients' quality of life scores, such as those associated with health-related quality of life (HRQOL), physical activity, and treatment satisfaction and health utility.

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A patient without inhibitors refers to a patient who does not have such alloantibodies. The present treatment methods are beneficial for hemophilia A patients with or without inhibitors, as well as for hemophilia B patients with or without inhibitors.

### I. Fitusiran Pharmaceutical Compositions

Hemophilia results in a profound defect in thrombin generation, and further, hemophilia severity is correlated with the inability to generate thrombin. Without being bound by theory, it is believed that fitusiran-mediated lowering of antithrombin (AT) levels will increase thrombin generation and thus improve hemostasis in patients with hemophilia. Antithrombin is encoded by the SERPINC1 gene.

Fitusiran, whose structure is described herein, is a synthetically, chemically modified double-stranded small interfering RNA (siRNA) oligonucleotide covalently linked to a tri-antennary N-acetyl-galactosamine (GalNAc) ligand targeting the AT3 mRNA in the liver, thereby suppressing the synthesis of antithrombin. The nucleosides in each strand of fitusiran are connected through either 3′-5′ phosphodiester or phosphorothioate linkages, thus forming the sugar-phosphate backbone of the oligonucleotide.

The sense strand and the antisense strand of fitusiran contain 21 and 23 nucleotides, respectively. The 3′-end of the sense strand is conjugated to the GalNAc containing moiety (referred to as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. Nos. 9,127,274, 11,091,759, and WO 2019/014187.

The two nucleotide strands of fitusiran are shown below:

sense strand:
(SEQ ID NO: 1)
5′Gf-ps-Gm-ps-Uf-Um-Af-Am-Cf-Am-Cf-Cf-Af-Um-Uf-Um-
Af-Cm-Uf-Um-Cf-Am-Af-L96 3′,
and
antisense strand:
(SEQ ID NO: 2)
5′ Um-ps-Uf-ps-Gm-Af-Am-Gf-Um-Af-Am-Af-Um-Gm-Gm-
Uf-Gm-Uf-Um-Af-Am-Cf-Cm-ps-Am-ps-Gm 3′,

wherein
Af=2′-fluoroadenosine (i.e., 2′-deoxy-2′-fluoroadenosine)
Cf=2′-fluorocytidine (i.e., 2′-deoxy-2′-fluorocytidine)
Gf=2′-fluoroguanosine (i.e., 2′-deoxy-2′-fluoroguanosine)
Uf=2′-fluorouridine (i.e., 2′-deoxy-2′-fluorouridine)
Am=2′-O-methyladenosine
Cm=2′-O-methylcytidine
Gm=2′-O-methylguanosine
Um=2′-O-methyluridine
“-” (hyphen)=3′-5′ phosphodiester linkage sodium salt
“-ps-”=3′-5′ phosphorothioate linkage sodium salt
   and wherein L96 has the following formula:

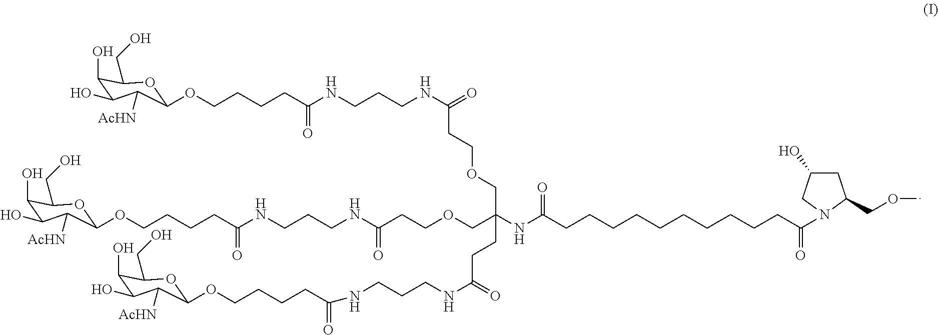

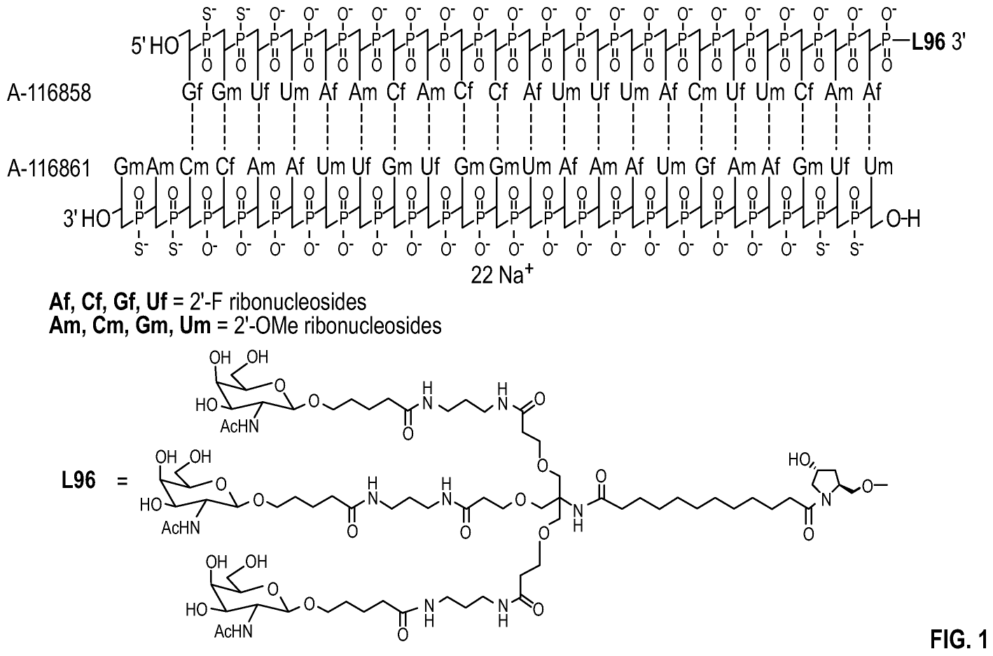

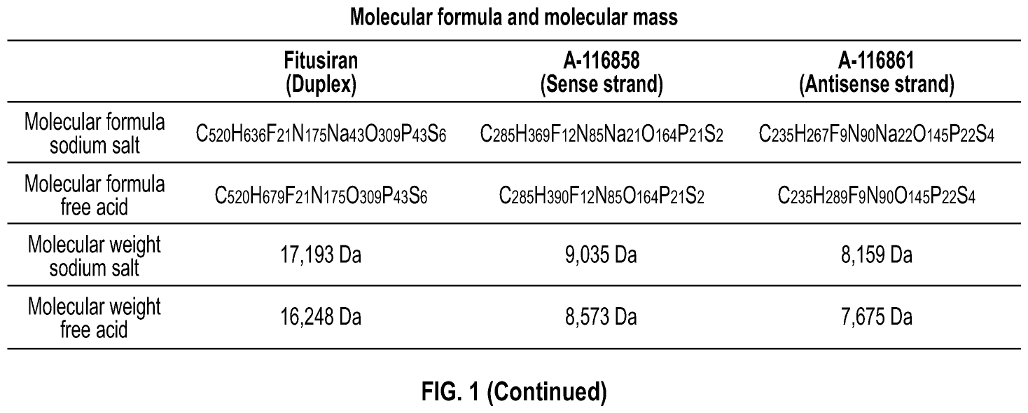

The expanded structural formula, molecular formula, and molecular weight of fitusiran (sodium form) are shown in FIG. 1.

The structure of fitusiran can also be described using the following diagram, wherein the X is O:

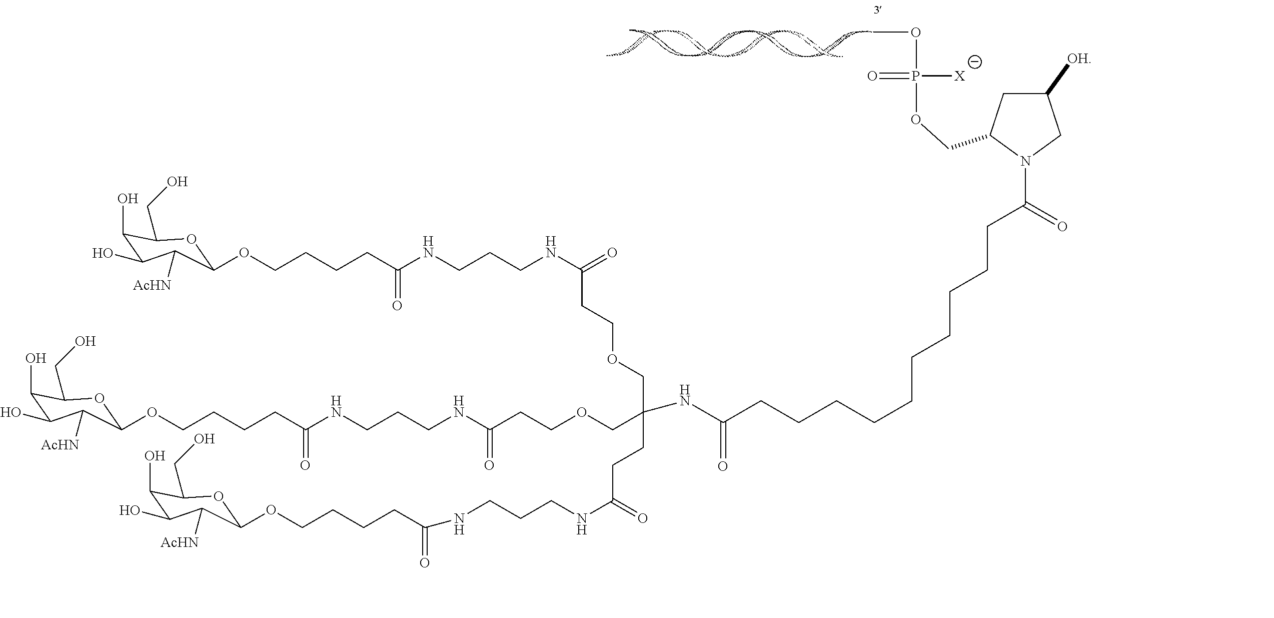

For use in the present treatment methods, fitusiran may be provided in a pharmaceutical composition comprising it and a pharmaceutically acceptable excipient. In certain embodiments, fitusiranis in sodium salt form.

In some embodiments, fitusiran is provided in an aqueous solution at a concentration of about 1 to about 200 mg/mL (e.g., about 50 to about 150 mg/mL, about 80 to about 110 mg/mL, about 90 to about 110 mg/mL, about 5 to about 25 mg/mL, about 7.5 to about 20 mg/mL, or about 10 to about 15 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical composition comprises fitusiran at a concentration of about 6.25, about 12.5, about 50, about 75, about 100, about 125, about 150, or about 200 mg/mL.

Unless otherwise indicated, a fitusiran weight recited in the present disclosure is the weight of fitusiran free acid (the active moiety), even though fitusiran is injected to patients subcutaneously in its sodium form (in an aqueous solution). For example, 100 mg/mL fitusiran means 100 mg of fitusiran free acid (equivalent to 106 mg fitusiran sodium, the drug substance) per mL.

In some embodiments, the pharmaceutical compositions comprise fitusiran in a phophate-buffered saline. The phosphate concentration in the solution may be about 1 to about 10 mM (e.g., about 2, about 3, about 4, about 5, about 6, about 7, about 8, or about 9 mM), with a pH of 6.0-8.0. The pharmaceutical compositions herein may include a preservative such as EDTA. Alternatively, the pharmaceutical compositions are preservative-free. In particular embodiments, the fitusiran pharmaceutical composition is preservative-free and comprises, consists of, or consists essentially of about 100 mg of fitusiran per mL of a 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the composition to 7.0.

The pharmaceutical composition may be provided in a container (e.g., a vial or a syringe). The container may contain single or multiple doses. In some embodiments, the container is a single-use container (e.g., a single-use ampule or a single-use syringe, such as a single-use pre-filled syringe), with each container containing about 10 to about 100 mg fitusiran (e.g., about 10 mg, about 20 mg, about 25 mg, about 40 mg, about 50 mg, or about 80 mg). The fitusiran may be provided in a solid form in the container and reconstituted in an aqueous solution (e.g., PBS) prior to use, with the reconstituted solution containing about 1 to about 150 mg/mL (e.g., about 6.25 mg/mL, about 12.5 mg/mL, or about 100 mg/mL) fitusiran. In some embodiments, fitusiran is provided in sodium salt form in a single-use glass vial or a single-use prefilled syringe (e.g., one with a safety system). In further embodiments, each vial or syringe contains about 80 mg of fitusiran in about 0.8 mL (or about 50 mg of fitusiran in about 0.5 mL, about 20 mg of fitusiran in about 0.2 mL, or about 10 mg of fitusiran in about 0.1 mL) of 5 mM phosphate buffered saline solution (pH 7.0); and the solution is administered to patients through subcutaneous injection. The solution can be stored at 2 to 30° C. (e.g., 2 to 8° C.).

In particular embodiments, the fitusiran composition for subcutaneous injection contains fitusiran in a 5 mM phosphate buffered saline having 0.64 mM NaH₂PO₄, 4.36 mM Na₂HPO₄, and 84 mM NaCl at pH 7.0. In certain embodiments, the composition of fitusiran solution for subcutaneous injection is shown in Table 1A below:

TABLE 1A
Exemplary Formulation

| | Composition | | | |
|---|---|---|---|---|
| | | | Per unit | Per unit |
| | | | (2 mL | (1 mL |
| | Percentage | Per ml | vial) | syringe) |
| Components | [%] | [mg] | [mg] | [mg] |
| Fitusiran (active moiety) | 10 | 100 [106] | 80 [84.8] | 80 [84.8] |
| [equivalent to fitusiran | | | | |
| sodium] | | | | |
| Sodium chloride | 0.49 | 4.909 | 3.927 | 3.927 |
| Dibasic sodium | 0.12 | 1.169 | 0.935 | 0.935 |
| phosphate (heptahydrate) | | | | |
| Monobasic sodium | <0.01 | 0.0885 | 0.0708 | 0.0708 |
| phosphate (monohydrate) | | | | |
| Phosphoric acid, | — | q.s. | q.s. | q.s. |
| concentrated | | pH 7.0 | pH 7.0 | pH 7.0 |
| Sodium hydroxide | — | q.s. | q.s. | q.s. |
| | | pH 7.0 | pH 7.0 | pH 7.0 |
| Water for injection | q.s. 100 | q.s. | q.s. | q.s. |
| | | 1 mL | 0.8 mL | 0.8 mL |

q.s.: quantum satis.

In certain embodiments, the composition of fitusiran solution for subcutaneous injection is shown in Table 1B below.

TABLE 1B
Fitusiran Formulations

| Components | Formulation 1 (mg) | Formulation 2 (mg) |
|---|---|---|
| Fitusiran | 12.5 [13.25] | 100 [106] |
| [Na salt] | | |
| NaH₂PO₄*H₂O | 0.388 | 0.0885 |
| Na₂HPO₄*7H₂O | 0.586 | 1.169 |
| NaCl | 8.7 | 4.909 |
| 0.1N NaOH | q.s. | q.s. |
| 0.1M H₃PO₄ | q.s. | q.s. |
| Purified water | Ad 1 mL | Ad 1 mL |

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH).

### II. Therapeutic Use of Fitusiran

Fitusiran can suppress liver production of antithrombin (AT). In its role as an anti-coagulant, AT regulates hemostasis by directly targeting thrombin production or by inactivating uncomplexed FXa, which in turn reduces thrombin production (Quinsey et al., Int J Biochem Cell Biol. (2004) 36(3):386-9). Fitusiran may be used to treat those who have impaired hemostasis.

For example, fitusiran can be used to treat hemophilia A or B with or without inhibitors in patients for routine prophylaxis to prevent or reduce the frequency of bleeding episodes. In particular embodiments, fitusiran is used to treat hemophilia A or B (congenital factor VIII or factor IX deficiency, respectively) with or without inhibitors in adult and adolescent patients (≥12 years of age).

The present methods include administering to the hemophilia patient (e.g., a hemophilia A or B patient) in need thereof a therapeutically effective amount of fitusiran. “Therapeutically effective amount” refers to the amount of fitusiran that helps the patient to achieve a desired clinical endpoint. A desired clinical endpoint may be, for example, reduction of annual bleeding rates (ABR) (e.g., by more than 10, 20, 30, 40, 50, 60, 70, 80, 90, or 100%). A desired clinical endpoint may be reduction of antithrombin levels in the patient to a normal level (e.g., about 64-210 nM), reduction in annualized bleeding rate (ABR), reduction of spontaneous bleeding rate, and improvement in patient Quality of Life (QoL).

A desired clinical endpoint may also be improved patient-reported outcomes (PROs) as further described below. In some embodiments, the treatment efficacy can be measured by a reduction in the signs and symptoms or severity of disease as evaluated by the patient based on a valid and reliable PRO instrument. Any positive change resulting in, for example, lessening of the signs and symptoms or severity of disease measured using the appropriate scale, represents adequate treatment using the pharmaceutical compositions as described herein.

Hemophilia, through its associated symptoms, functional limitations and treatment burden, directly impacts the health-related quality of life (HRQoL) of patients. Improving HRQOL is a critical aspect of hemophilia disease management. The present methods improve HRQOL of patients as determined by well-designed, detailed and validated questionnaires. For examples, HRQOL in adult patients (17 years or older, e.g., 18 years or older) can be measured by questionnaires Haem-A-QoL, EuroQol 5-Dimensions (EQ-5D-5L) (EuroQol Group, Health Policy (1990) 16(3):199-208), Hemophilia Activities List (HAL), and Treatment Satisfaction Questionnaire for Medication (TSQM-9). HRQoL in adolescent patients (12 years or older to 17 years old) can be measured by, e.g., Haemophilia Quality of Life Questionnaire for teenagers (Haemo-QoL) (See, e.g., Bullinger et al., Value Health. (2009) 12(5):808-20; and Remor, Int J Behav Med. (2013) 20(4):609-17) and the Pediatric Hemophilia Activities List (pedHAL).

In some embodiments, the present treatment improves the quality of life of patients in one or more of QoL domains (e.g., hemophilia-specific QoL domains). These QoL domains include, for example, domains related to Physical Health, Feeling, View of Self, Sports and Leisure, Work and School, Dealing with Hemophilia, Treatment, Future, Family Planning, and/or Partnership and Sexuality. Improvement in these domains may be evaluated by patient-reported outcome (PRO) and may be aided by questionnaires. For example, improvement in these domains may be reported by a patient to his/her physician, and/or may be scored by a QoL questionnaire.

The PRO instrument may be, for example, the Haemophilia Quality of Life Questionnaire for Adults (“Haem-A-QoL”; von Mackensen et al., Haematologica (2005) 90(s2):115-6, Abstract 0290; Wyrwich et al., Haemophilia (2015) 21(5):578-84). In particular embodiments, HRQoL of adult patients is measured by scores in Haem-A-QoL. See, e.g., von Mackensen et al., Value in Health. (2005) 8(6):A127; von Mackensen et al., J Thrombosis and Haemostasis. (2005) 3(Sup1):P0813; von Mackensen and Gringeri, “Quality of Life in Hemophilia” In: Handbook of Disease Burdens and Quality of Life Measures. Heidelberg: Springer; 2009, pp. 1910-1; and Bullinger et al., Value in Health. (2009) 12(5):808-20; Wyrwich, supra. The Haem-A-QoL questionnaire includes 46 items contributing to 10 domains, including Physical Health (5 items), Feeling (4 items), View of Self (5 items), Sports and Leisure (5 items), Work and School (4 items), Dealing with Hemophilia (3 items), Treatment (8 items), Future (5 items), Family Planning (4 items), and Partnership and Sexuality (3 items).

All Haem-A-QoL items are measured based on a 5-point frequency scale (1=never, 2=rarely, 3=sometimes, 4=often, and 5=all the time). A “Not Applicable” response option is also available for the domains of “Sports & Leisure,” “Work & School,” and “Family Planning” when the question does not apply to the participant. A “Total Score” is also used to represent the average of all 10 domains of the Haem-A-QoL questionnaire. Haem-A-QoL domain scores and the Total Score are transformed to a scale of 0-100 (using an instrument-specific scoring system) with higher scores representing greater impairment. A decrease in score relative to the corresponding baseline score (score before the treatment being evaluated) indicates an improvement in the patient's quality of life.

The present fitusiran therapy improves the score from at least one of the Haem-A-QoL domains (e.g., Physical Health, Feeling, View of Self, Sports and Leisure, Work and School, Dealing with Hemophilia, Treatment, Future, Family Planning, and/or Partnership and Sexuality) from baseline, and/or improves the Haem-A-QoL Total Score from baseline. In particular, the present methods may improve the quality of life of hemophilia patients, including improvement (e.g., alleviation and disappearance) of patient-reported hemophilia-related symptoms (e.g., painful swellings and joint pain) and physical functioning (e.g., pain with movement and difficulty walking far) as determined by the Physical Health score and/or the Total Score of Haem-A-QoL. A clinically meaningful improvement of quality of life includes, for example, an about 7 or more point reduction in the Total Score, an about 10 or more point reduction in the Sports and Leisure domain score, and/or an about 10 or more point reduction in the Physical Health domain score. See Wyrwich, supra. In some embodiments, one or more of the 10 domain scores (e.g., the Physical Health domain score or the Total Score) in Haem-A-QoL is reduced by 1 or more units (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more units).

For patients <17 years of age, Haemo-QoL, a shorter version of Haem-A-QoL specifically used for children and adolescents of age groups II/III (8-16 years of age), can be used to measure PROs. The Haemo-QoL survey has nine domains, including Physical Health, Feeling, View of Yourself, Dealing with Hemophilia, Treatment, Family, Friends, Others, and Sports. A “Total Score” is also used to represent the average of all nine domains of the Haemo-QoL questionnaire. Scoring for the Haemo-QoL instrument follows the same methodology described above for the Haem-A-QoL instrument. In some embodiments, one or more of the nine domain scores (e.g., the Physical Health domain score or the Total Score) in Haemo-QoL is reduced by 1 or more units (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more units).

The EQ-5D-5L is a standardized and disease-generic instrument for use as a measure of QoL outcome. It consists of a questionnaire pertaining to five dimensions (Mobility, Self-Care, Usual Activities, Pain/Discomfort, and Anxiety/Depression and a visual analog scale). Scoring of the questionnaire is based on five degrees of disability (none, slight, moderate, severe, or extreme). The EQ-5D-5L instrument additionally includes a stand-alone visual analog scale (VAS); scoring of the VAS ranges from 0 (worst imaginable health state) to 100 (best imaginable health state). Higher scores indicate better health status. See also e.g., Herdman et al., Qual Life Res (2011) 20(10):1727-36.

The present fitusiran therapy improves the score from at least one of the EQ-5D-5L dimensions (e.g., Mobility, Self-Care, Usual Activities, Pain/Discomfort, and Anxiety/Depression) from baseline. In some embodiments, one or more of the five dimension scores or the total score in the EQ-5D-5L is increased by 0.01 or more units (e.g., 0.02 or more, 0.03 or more, 0.04 or more, 0.05 or more, 0.06 or more, 0.07 or more, 0.08 or more, 0.9 or more, 0.10 or more, 0.11 or more, 0.12 or more, 0.13 or more, 0.14 or more, 0.15 or more, 0.16 or more, 0.17 or more, 0.18 or more, 0.19 or more, or 0.20 or more units). In some embodiments, the VAS score is increased by 1 or more units (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more units).

The present methods improve patient satisfaction with treatment as determined by well-designed, detailed questionnaires, including the Treatment Satisfaction Questionnaire for Medication (TSQM-9). The TSQM-9 is a validated psychometric tool that provides a general measure of patient satisfaction with medication. See also, e.g., Atkinson et al., Health Qual Life Outcomes (2004)2:12 and Bharmal et al., Health Qual Life Outcomes (2009)7:36. The TSQM-9 questionnaire includes nine items contributing to three domains, including Effectiveness, Convenience, and Global Satisfaction. TSQM-9 domain scores range from 0 to 100, with higher scores denoting improved HRQoL. An increase in score relative to the corresponding baseline score (score before the treatment being evaluated) indicates an improvement in the patient's satisfaction with treatment. The EQ-5D-5L and TSQM-9 surveys are administered to patients ≥18 years of age.

The present fitusiran therapy improves the score from at least one of the TSQM-9 domains (i.e., Effectiveness, Convenience, and Global Satisfaction) from baseline. In particular, the present methods may improve the treatment satisfaction of hemophilia patients. In some embodiments, one or more of the three domain scores (i.e., Effectiveness, Convenience, and Satisfaction) in TSQM-9 is increased by 1 or more units (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more units).

The present methods improve subjective functional ability to perform activities of daily living as determined by well-designed, detailed questionnaires, including the Hemophilia Activities List (HAL) and Pediatric Hemophilia Activities List (pedHAL).

The HAL is used to assess patients ≥18 years of age, and the pedHAL is used to assess patients <18 years of age. See also, e.g., van Genderen et al., Haemophilia (2006) 12(1):36-46. The HAL questionnaire includes 42 items contributing to ten domains, including Lying Down/Sitting/Kneeling/Standing, Functions of the Legs, Functions of the Arms, Use of Transportation, Self Care, Household Tasks, Leisure Activities and Sport, Basic Lower Extremity Activities, Upper Extremity Activities, and Complex Lower Extremity Activities. A “Total Score” is also used to represent the average of all ten domains of the HAL questionnaire. HAL domain scores range from 0 to 100, with higher scores representing higher self-perceived functional ability. An increase in score relative to the corresponding baseline score (score before the treatment being evaluated) indicates an improvement in the patient's self-perceived functional ability.

The pedHAL questionnaire includes 53 items contributing to seven domains, including Lying Down/Sitting/Kneeling/Standing, Functions of the Legs, Functions of the Arms, Use of Transportation, Self Care, Household Tasks, and Leisure Activities and Sport. Scoring for the pedHAL instrument follows the same methodology described above for the HAL instrument.

The present fitusiran therapy improves the score from at least one of the HAL or pedHAL domains (i.e., Lying Down/Sitting/Kneeling/Standing, Functions of the Legs, Functions of the Arms, Use of Transportation, Self Care, Household Tasks, Leisure Activities and Sport, Basic Lower Extremity Activities (HAL only), Upper Extremity Activities (HAL only), and Complex Lower Extremity Activities (HAL only)) from baseline. In particular, the present methods may improve the subjective functional ability to perform activities of daily living of hemophilia patients. In some embodiments, one or more of the domain scores in HAL or pedHAL is increased by 1 or more units (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more units).

For all questionnaires described above, the questionnaire may be taken before treatment and after treatment with one or more (e.g., two or more, three or more, four or more, five or more, or six or more) doses of fitusiran (e.g., administered subcutaneously at about 10, about 20, about 50, or about 80 mg about once every four weeks or about once a month or about once every eight weeks or about once every two months). For example, the questionnaire may be taken at week 8, 12, 16, 20, 24, 28, 32, 35, 36, 37, or 38 weeks after commencement of fitusiran treatment. The questionnaire may be taken approximately 253 days (e.g., 250, 251, 252, 253, 254, or 255 days) after commencement of fitusiran treatment.

In some embodiments, a patient with hemophilia A or B with or without inhibitors is treated with a subcutaneous dose of fitusiran at about 50 mg per dose about every two months (or about every eight weeks). In other embodiments, a patient with hemophilia A or B with or without inhibitors is treated with a subcutaneous dose of fitusiran at about 50 mg about every month (or about every four weeks). In yet other embodiments, a patient with hemophilia A or B with or without inhibitors is treated with a subcutaneous dose of fitusiran at about 80 mg about every two months (or about every eight weeks). In yet other embodiments, a patient with hemophilia A or B with or without inhibitors is treated with a subcutaneous dose of fitusiran at about 80 mg about every month (or about every four weeks). In yet other embodiments, a patient with hemophilia A or B with or without inhibitors is treated with a subcutaneous dose of fitusiran at about 20 mg about every two months (or about every eight weeks). In yet other embodiments, a patient with hemophilia A or B with or without inhibitors is treated with a subcutaneous dose of fitusiran at about 20 mg about every month (or about every four weeks). In yet other embodiments, a patient with hemophilia A or B with or without inhibitors is treated with a subcutaneous dose of fitusiran at about 10 mg about every month (or about every four weeks).

### III. Administration of Fitusiran Pharmaceutical Compositions

The fitusiran pharmaceutical composition may be administered by any means known in the art including, but not limited to, intraperitoneal, intravenous, intramuscular, subcutaneous, transdermal, or hepatic portal vein administration. In certain embodiments, the pharmaceutical composition is administered by subcutaneous injection at a dose strength of, for example, about 10 to about 100 mg (e.g., about 10 to about 90 mg, about 10 to about 80 mg, about 20 to about 80 mg, about 40 to about 90 mg, about 50 to about 100 mg, about 50 to about 90 mg, about 50 to about 85 mg, or about 50 to about 80 mg) per dose. In particular embodiments, fitusiran is administered subcutaneously at about 10, about 20, about 50 or about 80 mg (weight of active moiety) per dose in a PBS solution as described above.

A plurality of fitusiran doses may be administered to a subject at an interval of about 1, about 2, about 3, about 4, about 5, about 6, about 7, or about 8 weeks, or of about 1, about 2, or about 3 months. In particular embodiments, a fixed dose of fitusiran (e.g., about 10 mg, about 20 mg, about 50 mg, or about 80 mg subcutaneous injection) is administered to a hemophilia patient (e.g., a hemophilia A or hemophilia B patient who is optionally twelve years or older and who has or has not developed inhibitors) about once every four weeks or about once a month, or about once every eight weeks or about once every other month.

In some embodiments, the present pharmaceutical compositions can be administered with other pharmaceuticals and/or other therapeutic methods, such as those that are currently employed for treating bleeding disorders. For example, in certain embodiments, fitusiran is administered in combination with a second agent useful in treating hemophilia A and/or B. Examples of such second agents are fresh-frozen plasma (FFP); rFVIIa; aPCC; recombinant or plasma-derived FVIII or FIX; virus-inactivated, vWF-containing FVIII concentrates; desensitization therapy which may include large doses of FVIII or FIX, along with steroids or intravenous immunoglobulin (IVIG) and cyclophosphamide; plasmapheresis in conjunction with immunosuppression and infusion of FVIII or FIX, with or without antifibrinolytic therapy; immune tolerance induction (ITI), with or without immunosuppressive therapy (e.g., cyclophosphamide, prednisone, and/or anti-CD20); desmopressin acetate (DDAVP); antifibrinolytics, such as aminocaproic acid and tranexamic acid; antihemophilic agents; corticosteroids; immunosuppressive agents; and estrogens. The fitusiran composition and the additional therapeutic agent and/or treatment may be administered at the same time and/or in the same combination, e.g., parenterally, or the additional therapeutic agent can be administered as part of a separate composition or at separate times and/or by another method known in the art or described herein.

### IV. Articles of Manufacture and Kits

The present invention also provides kits comprising a pharmaceutical composition for use in the present treatment methods. Such kits include one or more vials or one or more pre-filled syringes comprising a pharmaceutical composition of the invention and instructions for use, e.g., instructions for administering a therapeutically effective amount of fitusiran. The kits may optionally further comprise means for administering fitusiran (e.g., an injection device), or means for measuring the inhibition of SERPINC1 (e.g., means for measuring the inhibition of SERPINC1 mRNA, expression of protein encoded by SERPINC1, and/or SERPINC1 activity). Such means for measuring the inhibition of SERPINC1 may comprise a means for obtaining a sample from a subject, such as, e.g., a plasma sample. The kits of the invention may optionally further comprise means for determining the therapeutically effective or prophylactically effective amount.

Additional definitions of terminology and exemplary embodiments are described in the Examples and are incorporated by reference herein.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of hematology, medicine, medicinal and pharmaceutical chemistry, and cell biology described herein are those well-known and commonly used in the art. Further, unless otherwise required by context, singular terms shall include pluralities and plural terms shall include the singular. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain aspects, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Clinical Study Design and Population

This Example describes the Phase 3 clinical trials protocols used in studies of fitusiran administered subcutaneously to male patients having severe hemophilia A or B with or without inhibitors.

### Study Assessments

Patient-reported outcomes were utilized to assess health-related quality of life (HRQOL), physical activity, and treatment satisfaction and health utility. The Haem-A-QoL was provided to patients ≥17 years of age. The Haemo-QoL (children's and adolescent's short version for age groups II/III [8-16 years of age]) was provided to patients <17 years of age. The Treatment Satisfaction Questionnaire for Medication (TSQM) was used to assess patient satisfaction with treatment in patients ≥18 years of age. The HAL was assessed in patients ≥18 years of age, and the pedHAL will be assessed in patients <18 years of age. The EQ-5D-5L was used as a measure of QOL outcome in patients ≥18 years of age.

### Study Design

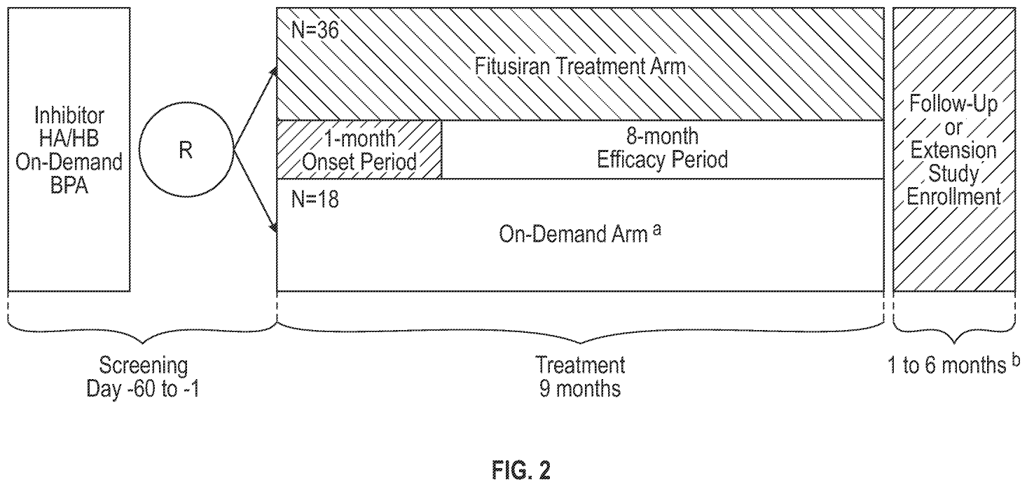

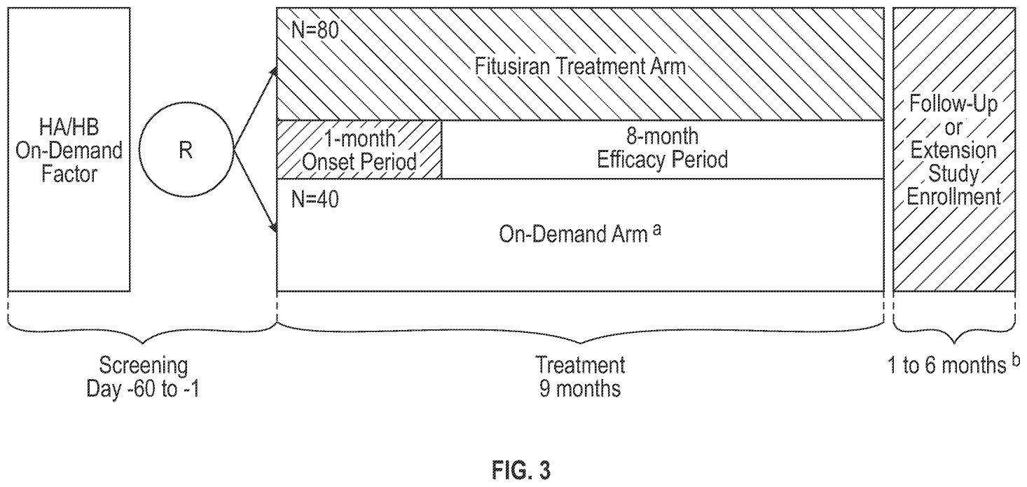

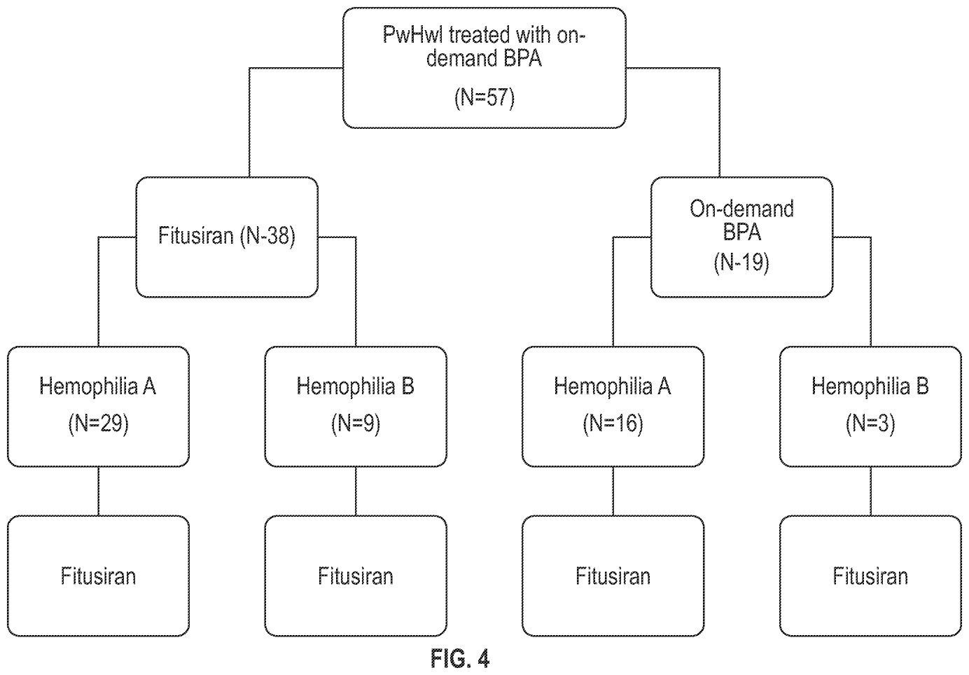

The clinical trials described herein are open-label Phase 3 studies designed to evaluate the efficacy and safety of fitusiran in male patients aged ≥12 years with hemophilia A or B, with or without inhibitory antibodies to FVIII or FIX, who are not receiving prophylactic therapy. The study designs are described in FIG. 2 (patients with inhibitors) and FIG. 3 (patients without inhibitors). FIG. 4 describes the participant disposition of patients enrolled in the Phase 3 clinical trial of fitusiran in Hemophilia A and B patients with inhibitors.

Eligible patients were randomized in a 2:1 ratio to the following arms:
i. Fitusiran treatment arm: fitusiran 80 mg administered SC as prophylaxis once monthly, with use of on-demand BPA or factor concentrate for treatment of breakthrough bleeding episodes, and
ii. On-demand arm: on-demand (OD) use of BPA or factor concentrate for treatment of breakthrough bleeding episodes.

On-demand use of BPA or factor concentrate is defined as the use of these agents, as needed, for episodic bleeding episodes, and not on a regular regimen intended to prevent spontaneous bleeding. Throughout the study, patients in the fitusiran treatment arm received on-demand treatment for breakthrough bleeding episodes with BPA or factor concentrate, as appropriate. In the clinical trials, factor concentrate is also referred to as clotting factor concentrate (CFC).

All patients were treated for a total of nine months; patients randomized to the fitusiran treatment arm received a total of nine subcutaneous injections of fitusiran. Because the full PD effect of fitusiran is not achieved until approximately 28 days after receiving the first dose, efficacy was assessed during the final eight months on study (Day 29 to Month 9). Therefore, the overall fitusiran treatment period was defined as the onset period (Day 1 to Day 28 after receipt of the first dose, during which the AT lowering capacity of fitusiran is increasing but has not yet reached therapeutic levels) plus the efficacy period (Day 29 and after, when the AT lowering capacity of fitusiran has achieved therapeutic target range).

Study population

The inclusion criteria were as follows:
i. males ≥12 years of age,
ii. severe hemophilia A or B with or without inhibitors evidenced by a central laboratory measurement or documented medical record evidence of FVIII <1% or FIX level ≤2% prior to treatment, and
iii. a minimum of six bleeding episodes requiring BPA or factor concentrate treatment within the last six months prior to treatment.

A patient with severe hemophilia A or B with inhibitors is evidenced by on-demand use of BPAs to manage bleeding episodes for at least the last six months prior to treatment, and must meet at least one of the following Nijmegen-modified Bethesda assay results criteria:
i. inhibitor titer of ≥0.6 BU/mL prior to treatment, or
ii. inhibitor titer of <0.6 BU/mL prior to treatment with medical record evidence of two consecutive titers ≥0.6 BU/mL, or
iii. inhibitor titer of <0.6 BU/mL prior to treatment with medical record evidence of anamnestic response.

A patient with severe hemophilia A or B without inhibitors is evidenced by on-demand use of factor concentrate to manage bleeding episodes for at least the last six months prior to treatment, and must meet each of the following criterion:
i. Nijmegen modified Bethesda assay inhibitor titer of <0.6 BU/mL,
ii. no use of BPAs to treat bleeding episodes for at least the last six months, and
iii. no history of immune tolerance induction therapy within the last six months.

The key exclusion criteria were as follows:
i. known co-existing bleeding disorders other than hemophilia A or B, i.e., Von Willebrand's disease, additional factor deficiencies, or platelet disorders,
ii. current use of BPA (for patients with inhibitors) or factor concentrates (for patients without inhibitors) as regularly administered prophylaxis designed to prevent spontaneous bleeding episodes,
iii. AT activity <60% prior to treatment,
iv. presence of clinically significant liver disease, as indicated by any of the conditions below:
   1. INR >1.2,
   2. ALT and/or AST >1.5×upper limit of normal reference range (ULN),
   3. total bilirubin >ULN (>1.5×ULN in patients with Gilbert's Syndrome),
   4. history of portal hypertension, esophageal varices, or hepatic encephalopathy, and/or
   5. presence of ascites by physical exam,
v. hepatitis C virus antibody positive, except patients with a history of HCV infection who both:
   1. completed curative treatment at least 12 weeks prior to enrollment and attained sustained virologic response as documented by a negative HCV RNA at screening, or they have spontaneously cleared infection as documented by negative HCV RNA at Screening, and
   2. have no evidence of cirrhosis according to one of the following assessments:
      a. FibroScan <12.5 kPa (where available), or
      b. FibroTest score <0.75 and APRI <2 (if FibroScan unavailable),
vi. presence of acute hepatitis, i.e., hepatitis A or hepatitis E,
vii. presence of acute or chronic hepatitis B infection (IgM anti-HBC antibody positive or HBs Ag positive),
viii. platelet count ≤100,000/μL,
ix. presence of acute infection prior to treatment,
x. known to be HIV positive with CD4 count <200 cells/μL,
xi. estimated glomerular filtration rate ≤45 mL/min/1.73 m² (using the Modification of Diet in Renal Disease [MDRD] formula),
xii. co-existing thrombophilic disorder, as determined by presence of any of:
   1. FV Leiden mutation (homozygous or heterozygous),
   2. protein S deficiency,
   3. protein C deficiency, or
   4. prothrombin mutation (G20210A; homozygous or heterozygous),
xiii. history of antiphospholipid antibody syndrome,
xiv. history of arterial or venous thromboembolism, atrial fibrillation, significant valvular disease, myocardial infarction, angina, transient ischemic attack, or stroke (except patients who have experienced thrombosis associated with indwelling venous access),
xv. malignancy within two years prior to treatment, except for basal or squamous cell carcinoma of the skin that has been successfully treated,
xvi. significant active and poorly controlled (unstable) cardiovascular, neurologic, gastrointestinal, endocrine, renal or psychiatric disorders unrelated to hemophilia identified by key laboratory abnormalities or medical history,
xvii. anticipated need of surgery during the study or planned surgery scheduled to occur during the study,
xviii. completion of a surgical procedure within 14 days prior to treatment, or currently receiving additional BPA infusion for postoperative hemostasis,
xix. history of multiple drug allergies or history of allergic reaction to an oligonucleotide or GalNAc,
xx. inadequate venous access to allow the blood draws required by the study protocol,
xxi. history of intolerance to SC injection(s),
xxii. current or future participation in another clinical study, scheduled to occur during this study, involving an investigational product other than fitusiran or an investigational device; in order to participate in this study, patient must discontinue the investigational product or investigational device at least 30 days (or 5×the investigational product half-life, whichever is longer) prior to dosing (Day 1),
xxiii. current or prior participation in a gene therapy trial, and
xxiv. history of alcohol abuse within the 12 months prior to treatment. Alcohol abuse is defined as regular weekly intake of more than 14 units (unit: 1 glass of wine [approximately 125 mL]=1 measure of spirits (approximately 1 fluid ounce)=½pint of beer [approximately 284 mL]).

The baseline demographics of patients treated in each study are shown in Table 2. The baseline demographics are for the intent-to-treat (ITT) analysis set (i.e., all randomized patients). Overall, baseline patient characteristics where similar across groups.

TABLE 2
Mean Baseline Patient Demographics by Study (ITT Set)

| | Patients With Inhibitors | | | | Patients Without Inhibitors | | | | | |
|---|---|---|---|---|---|---|---|---|---|---|
| | OD/BPA | | Fitusiran | | OD/CFC | | Fitusiran | | | |
| | Arm | | Arm | | Arm | | Arm | | Overall | |
| Description | N = 19 | | N = 41 | | N = 40 | | N = 80 | | (N = 120) | |
| Age, years (SD) | 31.5 | (13.1) | 26.2 | (9.9) | 33.6 | (13.6) | 33.9 | (14.6) | 33.8 | (14.2) |
| Baseline Weight, kg | 70.2 | (17.6) | 66.1 | (18.5) | 75.3 | (17.3) | 73.0 | (19.1) | 73.8 | (18.5) |
| (SD) | | | | | | | | | | |
| Hemophilia A | 16 | (84.2%) | 32 | (78%) | 31 | (77.5%) | 62 | (77.5%) | 93 | (77.5) |
| n (%) | | | | | | | | | | |
| Hemophilia B | 3 | (15.8%) | 9 | (22%) | 9 | (22.5%) | 18 | (22.5%) | 27 | (22.5) |
| n (%) | | | | | | | | | | |
| Number of bleeding | 12.3 | (8.2) | 13.4 | (8.3) | 11.8 | (5.9) | 13.1 | (7.5) | 12.7 | (7.0) |
| episodes in the last six | | | | | | | | | | |
| months prior to start of | | | | | | | | | | |
| treatment (SD) | | | | | | | | | | |

### Dosing Regimens and Formulation

Fitusiran (SAR439774) solution for injection (SC use) was supplied as a sterile solution. Patients randomized to the fitusiran treatment arm received open-label 80 mg fitusiran as an SC injection once monthly, for a total of nine months.

### Example 2: Improvement of Patient Reported Outcomes by Fitusiran Treatment

An objective of the Phase 3 clinical trial studies of fitusiran was to evaluate patient-reported outcomes (PROs) to assess health-related quality of life (HRQOL), physical activity, and perception of treatment. The age of the patient at randomization determined which age-specific questionnaires was utilized, which remain in force for the full study duration.

In the studies, PROs were collected using:
Hemophilia Quality of Life Questionnaire for Adults (Haem-A-QoL);
Hemophilia Quality of Life Questionnaire for Children (Haemo-QoL);
Hemophilia Activity List (HAL);
Pediatric Hemophilia Activity List (pedHAL);
Treatment Satisfaction Questionnaire for Medication version 9 (TSQM-9); and
EuroQol 5-Dimensions, 5-Level (EQ-5D-5L) questionnaire.

The following sections describe the characteristics and results of each instrument. All patients were evaluated. For each questionnaire discussed in this Example, reported changes are from baseline to Day 253, which is also referred to as end of study (EOS).

### Hemophilia Quality of Life Questionnaire for Adults (Haem-A-QoL)

The Haem-A-QoL was provided to participants ≥17 years of age. Scoring for each item is based on a five-point Likert scale (never, rarely, sometimes, often, and all the time), and higher scores represent greater impairment. Negative values denote health-related quality of life (HRQoL) improvement. Changes from baseline to Day 253 in Haem-A-Qol by domain are reported in Table 3 below.

TABLE 3
Mean Changes in Haem-A-QoL by Domain and Study

| | Patients With Inhibitors | | | | Patients Without Inhibitors | | | |
|---|---|---|---|---|---|---|---|---|
| | OD/BPA | | Fitusiran | | OD/CFC | | Fitusiran | |
| | Arm | | arm | | Arm | | Arm | |
| Haem-A-QoL | N = 18 | | N = 32 | | N = 38 | | N = 72 | |
| Domains | Mean | SD | Mean | SD | Mean | SD | Mean | SD |
| Physical Health | −7.65 | 10.02 | −27.66 | 28.88 | −3.82 | 15.18 | −22.97 | 21.6 |
| Feeling | −5.88 | 23.01 | −17.74 | 29.41 | −3.13 | 17.83 | −11.87 | 22.02 |
| View of Yourself | −0.88 | 18.22 | −13.06 | 19.05 | −2.06 | 16.43 | −5.87 | 16.56 |
| Sports and Leisure | −0.74 | 16.62 | −17.43 | 22.89 | 1.43 | 14.47 | −8.63 | 19.91 |
| Work and School | −7.36 | 17.62 | −14.22 | 28.29 | −3.97 | 15.63 | −5.21 | 19.31 |
| Dealing with | −0.98 | 14.7 | −1.88 | 21.38 | −3.19 | 17.41 | −0.6 | 24.06 |
| Hemophilia | | | | | | | | |
| Treatment | 0 | 18.78 | −9.68 | 20.42 | −2.76 | 15.3 | −11.78 | 27.78 |
| Future | −0.59 | 15.3 | −10 | 21.49 | −4.26 | 13.44 | −10.29 | 17.15 |
| Family Planning | −10.23 | 16.7 | −8.48 | 17.6 | −6.58 | 23.78 | −7.01 | 20 |
| Partnership and | 2.94 | 26.34 | −12.63 | 25.81 | −4.9 | 11.98 | −2.69 | 25.94 |
| Sexuality | | | | | | | | |
| Total Score | −2.91 | 9.65 | −14.05 | 15.03 | −3.32 | 8.47 | −9.7 | 12.64 |

The Physical Health domain score showed a clinically meaningful improvement relative to baseline in the fitusiran prophylaxis arm in both the study of patients with inhibitors and the study of patients without inhibitors. Fitusiran-treated participants achieved comparatively improved physical health (painful swelling, joint pain, pain with movement, difficulty walking, time to get ready), with mean estimates suggesting that inhibitor patients benefit slightly more than non-inhibitors patients.

Total scores of Haem-A-QoL likewise demonstrated overall improvements in perceived QoL in the fitusiran prophylaxis arm, with clinically meaningful mean reductions from baseline reported in the fitusiran prophylaxis arms. Both inhibitor and non-inhibitor patients benefit from treatment with fitusiran, with inhibitor patients slightly outperforming in terms of improvement from baseline.

Additional analyses of Haem-A-Qol (secondary endpoint) were performed in the total score and physical health domain using an analysis covariance model (ANCOVA) which controls for potential study confounders. The results of these additional analyses are presented in Table 4.

TABLE 4
Mean Changes in Haem-A-QoL Physical Health and Total Score

| | Patients With Inhibitors | | | | Patients Without Inhibitors | | | |
|---|---|---|---|---|---|---|---|---|
| | OD/BPA | | Fitusiran | | OD/CFC | | Fitusiran | |
| | Arm | | Arm | | Arm | | Arm | |
| Haem-A-QoL | N = 18 | | N = 32 | | N = 38 | | N = 72 | |
| Domains | Mean | 95% CI | Mean | 95% CI | Mean | 95% CI | Mean | 95% CI |
| Total Score | −0.42 | −5.65; 4.8 | −15.27 | −19.3; −11.24 | −2.62 | −6.27; 1.03 | −9.68 | −12.51; −6.86 |
| Physical | −1.94 | −10.32; 6.43 | −30.67 | −36.9; −24.43 | −3.32 | −9.67; 3.04 | −23.07 | −28.00; 18.14 |
| Health | | | | | | | | |

The physical health domain score showed a significantly meaningful improvement relative to baseline in the fitusiran prophylaxis arm in both studies, regardless of analysis method (simple and ANCOVA based). Fitusiran-treated participants achieved comparatively improved physical health (painful swelling, joint pain, pain with movement, difficulty walking, time to get ready), with mean estimates suggesting that inhibitor patients benefit slightly more than non-inhibitors ones.

Total scores of Haem-A-QoL likewise demonstrated overall improvements in perceived QoL in the fitusiran prophylaxis arm, with reported mean significant reductions from baseline in the fitusiran prophylaxis regardless of analysis method (i.e., simple or ANCOVA based). Both inhibitor and non-inhibitor patients displayed benefit from treatment with fitusiran, with inhibitor patients slightly outperforming in terms of improvement from baseline.

### Hemophilia Quality of Life Questionnaire for Children (Haemo-QoL)

The Haemo-QoL questionnaires is a psychometrically tested QoL assessment instruments for participants with hemophilia aged ≤17 years. The Haemo-QoL questionnaires and proxy-versions are adapted from the original adult Haem-A-QoL for adolescents and children. Negative values denote hemophilia-related quality of life (HRQoL) improvement. Results of Haemo-QoL by domain and study are presented in Table 5.

TABLE 5
Mean Changes in Haemo-QoL by Domain and Study

| | Patients With Inhibitors | | | | Patients Without Inhibitors | | | |
|---|---|---|---|---|---|---|---|---|
| | OD/BPA | | Fitusiran | | OD/CFC | | Fitusiran | |
| | Arm | | Arm | | Arm | | Arm | |
| Haemo-QoL | N = 1 | | N = 6 | | N = 2 | | N = 8 | |
| Domains | Mean | SD | Mean | SD | Mean | SD | Mean | SD |
| Physical | −31.25 | NC | −32.64 | 40.07 | −3.13 | 30.94 | −21.09 | 28.53 |
| Health | | | | | | | | |
| Feeling | −6.25 | NC | −27.08 | 23.61 | 28.13 | 4.42 | −19.53 | 29.59 |
| View of | 37.5 | NC | −25 | 22.01 | 6.25 | 8.84 | −14.84 | 17.66 |
| Yourself | | | | | | | | |
| Dealing with | −6.25 | NC | −23.44 | 35.49 | 0 | 0 | −0.78 | 30.52 |
| Hemophilia | | | | | | | | |
| Treatment | −25 | NC | −40.63 | 31.25 | 12.5 | 0 | −14.06 | 28.3 |
| Family | 0 | NC | −14.06 | 10.67 | 3.13 | 22.1 | −18.75 | 19.48 |
| Friends | 50 | NC | 14.58 | 59.85 | 0 | 82.5 | 14.58 | 36.93 |
| Others | −25 | NC | −4.69 | 13.86 | −21.88 | 13.26 | −18.75 | 16.02 |
| Sports | 0 | NC | −31.6 | 32.21 | 12.5 | 70.71 | 2.34 | 22.14 |
| Total Score | −2.14 | NC | −18.21 | 4.23 | 4.29 | 16.16 | −10.8 | 12.83 |

Fitusiran patients generally reported improvements in all domains, with the exception of the domain of Friends in the study of patients with inhibitors and the domains of Friends and Sports in the study of patients without inhibitors. In both cases, total scores of Haemo-QoL displayed improvements in patients who received fitusiran.

### Hemophilia Activity List (HAL)

HAL scores assess subjective functional ability to perform activities of daily living (e.g., activities involving the upper and lower extremities, basic activities involving the lower extremities, and complex activities involving the lower extremities). Scores range from 0 to 100, with higher scores denoting fewer functional limitations. Results of HAL by domain and study are presented in Table 6.

TABLE 6
Mean Changes in HAL by Domain and Study

| | Patients with Inhibitors | | | | Patients Without Inhibitors | | | |
|---|---|---|---|---|---|---|---|---|
| | OD/BPA | | Fitusiran | | OD/CFC | | Fitusiran | |
| | Arm | | Arm | | Arm | | Arm | |
| | N = 18 | | N = 29 | | N = 35 | | N = 71 | |
| HAL Domains | Mean | SD | Mean | SD | Mean | SD | Mean | SD |
| Lying Down/Sitting/ | 3.24 | 18 | 8.53 | 19.7 | 1.95 | 9.93 | 7.57 | 18.27 |
| Kneeling/Standing | | | | | | | | |
| Functions of the Legs | 0.92 | 16.69 | 16.25 | 20.9 | 4.17 | 14.31 | 7.81 | 16.17 |
| Functions of the Arms | 3.24 | 20 | 13.79 | 23.17 | −0.94 | 15.68 | 11.47 | 19.41 |
| Use of Transportation | −10.83 | 21.62 | 13.56 | 26 | 1.15 | 21.93 | 10.83 | 24.25 |
| Self Care | 3.76 | 18.3 | 7.59 | 16.98 | 1.5 | 11.84 | 6.88 | 15.58 |
| Household Tasks | −5.02 | 16.66 | 17.26 | 21.15 | 2.82 | 13.85 | 10.84 | 18.06 |
| Leisure Activities and | −2.17 | 17.5 | 16.37 | 27.16 | 5.52 | 16.75 | 11.88 | 16.42 |
| Sport | | | | | | | | |
| Basic Lower Extremity | 1.18 | 21.57 | 18.28 | 18.74 | 4.69 | 18.55 | 8.28 | 17.5 |
| Activities | | | | | | | | |
| Upper Extremity | 3.53 | 15.26 | 10.34 | 17.31 | 0.42 | 11.34 | 8.92 | 16.09 |
| Activities | | | | | | | | |
| Complex Lower | 2.68 | 14.63 | 9.97 | 22.35 | 1.52 | 8.51 | 7.94 | 17.3 |
| Extremity Activities | | | | | | | | |
| Total Score | 0.27 | 9.16 | 13.74 | 16.4 | 2.46 | 9.32 | 9.06 | 13.03 |

Total mean scores for HAL (change from baseline to EOS) demonstrate that fitusiran prophylaxis patients show improvement in the total score and consistently across all ten domains in both studies. Higher observed improvements from the study of patients with inhibitors suggests that patients with inhibitors benefit most from fitusiran treatment.

Pediatric Hemophilia Activity List (pedHAL)

Children and adolescent specific pedHAL scores were calculated for participants below 18 years (Table 7).

TABLE 7
Mean Changes in pedHAL by Domain and Study

| | Patients With Inhibitors | | | | Patients Without Inhibitors | | | |
|---|---|---|---|---|---|---|---|---|
| | OD/BPA | | Fitusiran | | OD/CFC | | Fitusiran | |
| | Arm | | Arm | | Arm | | Arm | |
| | N = 1 | | N = 9 | | N = 5 | | N = 9 | |
| pedHAL Domains | Mean | SD | Mean | SD | Mean | SD | Mean | SD |
| Lying Down/Sitting/ | −42 | NC | 19.36 | 18.37 | −2.88 | 25.57 | 23.63 | 20.45 |
| Kneeling/Standing | | | | | | | | |
| Functions of the Legs | −60 | NC | 25.86 | 19.78 | −21.5 | 16.88 | 22.93 | 29.9 |
| Functions of the Arms | −56.67 | NC | 1.25 | 18.93 | −15 | 22.53 | 17.5 | 27.53 |
| Use of Transportation | −33.33 | NC | 12.08 | 14.02 | −1.67 | 20.64 | 28.33 | 31.22 |
| Self Care | −24.44 | NC | 8.09 | 12.61 | −1.67 | 19.99 | 22.12 | 27.22 |
| Household Tasks | −60 | NC | 30 | 22.61 | −17.5 | 23.63 | 25.71 | 36.19 |
| Leisure Activities and | −63.64 | NC | 25.82 | 17.44 | −3.27 | 33.39 | 38.28 | 32.67 |
| Sport | | | | | | | | |
| Total Score | −49.43 | NC | 18.87 | 16.68 | −9.5 | 21.65 | 25.61 | 26.14 |

For the total score and across each individual instrument domain, fitusiran prophylaxis displayed favorable results in patients with or without inhibitors, as seen by the change from baseline to EOS.

### Treatment Satisfaction Questionnaire for Medication Version 9

The studies of patients with hemophilia A or B with and without inhibitors also evaluated participant-reported outcome according to the Treatment Satisfaction Questionnaire for Medication Version 9 (TSQM-9). The TSQM-9 is a validated psychometric tool that provides a general measure of patient-reported perception of medication, and covers the domains Effectiveness, Convenience, and Global Satisfaction. Domain scores are ranged from 0 to 100, with higher scores denoting improved HRQOL. Results of TSQM-9 by domain and study are presented in Table 8.

TABLE 8
Mean Changes in TSQM-9 by Domain and Study

| | Patients With Inhibitors | | | | Patients Without Inhibitors | | | |
|---|---|---|---|---|---|---|---|---|
| | OD/BPA | | Fitusiran | | OD/CFC | | Fitusiran | |
| | Arm | | Arm | | Arm | | Arm | |
| TSQM-9 | N = 19 | | N = 38 | | N = 40 | | N = 80 | |
| Domains | Mean | SD | Mean | SD | Mean | SD | Mean | SD |
| Effectiveness | 6.21 | 21.6 | 21.11 | 23.68 | 0.49 | 25.78 | 16.43 | 20.36 |
| Convenience | 4.58 | 14.06 | 13.73 | 21.64 | 3.59 | 27.45 | 18.65 | 21.57 |
| Satisfaction | 3.36 | 17.33 | 19.12 | 23.9 | −0.42 | 20.35 | 11.2 | 19.87 |

Overall results of the mean TSQM-9 scores (change from baseline to EOS) consistently showed improvements for fitusiran in all three domains. Here again, improvements in inhibitor patients suggest that patients with inhibitors most benefit from fitusiran treatment.

### EuroQol 5-Dimension, 5-Level (EQ-5D-5L) and EuroQoL, Visual Analog Scale (EQ-VAS)

The EQ-5D-5L is a standardized and disease-generic instrument for use as a measure of QoL outcome. It consists of a questionnaire pertaining to five dimensions (mobility, self-care, usual activities, pain/discomfort, and anxiety/depression). Scoring of the questionnaire is based on five degrees of disability (none, slight, moderate, severe, or extreme) with a 0 (worst imaginable health state) to 1 (better health state) scale. Higher scores indicate better health status. EQ VAS is a continuous score ranging from 0 to 100. Scoring of the visual analog scale is based on a visual scale ranging from 0 (worst imaginable health state) to 100 (best imaginable health state). Results of EQ-5D-5L total score and VAS score are presented in Table 9.

TABLE 9
Mean Changes in EQ-5D-5L and EQ VAS by Study

| | Patients With Inhibitors | | | | Patients Without Inhibitors | | | |
|---|---|---|---|---|---|---|---|---|
| | OD/BPA | | Fitusiran | | OD/CFC | | Fitusiran | |
| | Arm | | Arm | | Arm | | Arm | |
| | N = 19 | | N = 38 | | N = 40 | | N = 80 | |
| EQ-5D-5L | Mean | SD | Mean | SD | Mean | SD | Mean | SD |
| EQ-5D-5L | 0.04 | 0.16 | 0.13 | 0.18 | 0 | 0.13 | 0.06 | 0.12 |
| Total Score | | | | | | | | |
| VAS Score | 5.22 | 17.09 | 14.39 | 19.64 | −0.08 | 15.67 | 8.12 | 16.99 |

Results from the EQ-5D-5L and EQ VAS denote in both studies an improvement from baseline, with notable changes in patients with inhibitors.

### Exit Interviews

The aim of the exit interview analysis was to better understand patients' and caregivers' experiences with hemophilia A or B (with or without inhibitors), and its treatments, including fitusiran, and how this experience evolved over the clinical trial period, as well as their perception and satisfaction with the treatment they received during the trials.

Patients were invited to participate in semi-structured exit interviews to share their experiences. Caregivers of patients were invited to participate in the interview if patients were unable (e.g., if the patients were under 18 years of age, or were cognitively unable). A total of 24 individuals were invited to participate in a 60-minute telephone interview. Following a semi-structured interview guide to ensure consistency across the interviews, participants were asked to describe their experiences with hemophilia and its treatment prior to entering any of the fitusiran trials. Next, participants were asked about their perceptions of the impact of hemophilia and its treatment on their daily lives, their treatment expectations, and experiences with fitusiran during the clinical trials. Participants were asked to rate different aspects of hemophilia treatments (improving joint health, decreasing bleeds, protection from bleed for entire month, improving joint mobility, prevention of fear/anxiety of bleeds, minimize anxiety/stress in managing hemophilia, and convenience) as “moderately important,” “very important,” or “extremely important.”

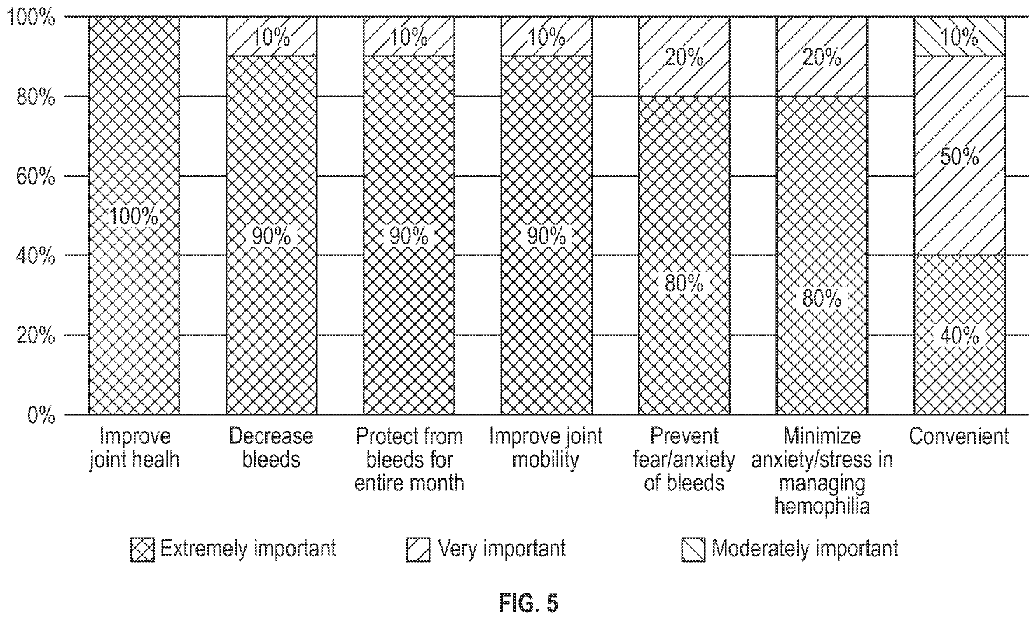

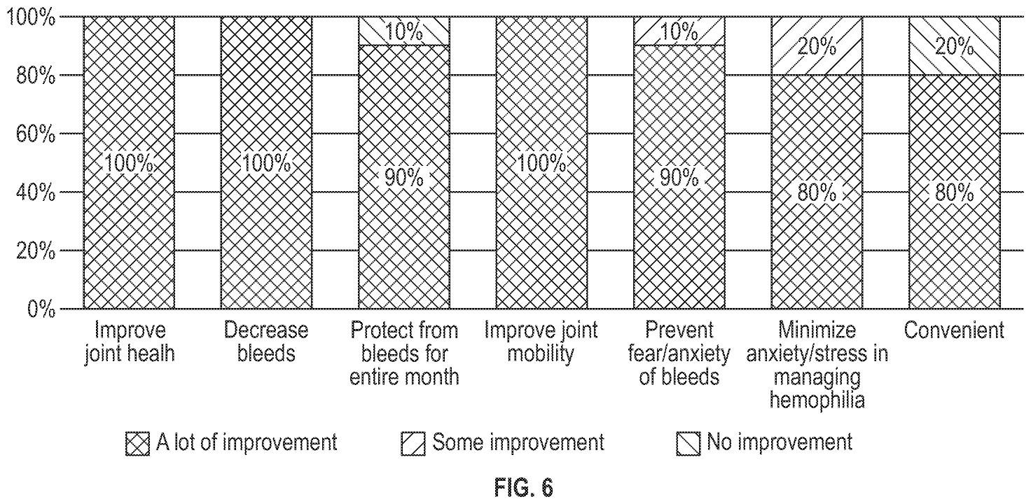

The data presented in FIGS. 5 and 6 reflect an interim analysis conducted on the first ten interviews. Eight patients with hemophilia A or B and two caregivers were interviewed. All participants rated improving joint health as extremely important; most participants rated decreasing bleeds (90%), protection from bleeds for an entire month (90%), improving joint mobility (90%), prevention of fear/anxiety of bleeds (80%) and minimizing anxiety/stress in managing hemophilia (80%) as “extremely important” aspects in their hemophilia treatment (FIG. 5). All participants reported improvements in these treatment aspects while under fitusiran prophylaxis. In particular, 100%, 100%, 90%, 100%, 90% and 80% of patients rated having “a lot of improvement,” respectively, in each of these aspects (FIG. 6). Almost all participants (n=9) preferred fitusiran prophylaxis over their previous hemophilia treatment and all reported being “very satisfied” with fitusiran treatment, especially with regards to its ability to prevent bleeds, durability of effects, and convenience. Among quotes recorded during the interviews, patients with hemophilia A or B reported, for example, that “after I took fitusiran, I became like a normal person” and that they felt “. . . very confident because if I get the fitusiran I can say no bleed will happen.”

These exit interviews provide encouraging and positive interim insights into the experiences of patients with hemophilia A or B, with or without inhibitors, and their caregivers. Prophylactic treatment with fitusiran demonstrated added value in patients with hemophilia A or B, irrespective of inhibitor status, particularly resulting in improving joint health, decreasing bleeds, and improving joint mobility and overall reducing their treatment burden.

### Example 3: Improvement of Additional Clinical Endpoints by Fitusiran Treatment

### Antithrombin Levels and Thrombin Generation

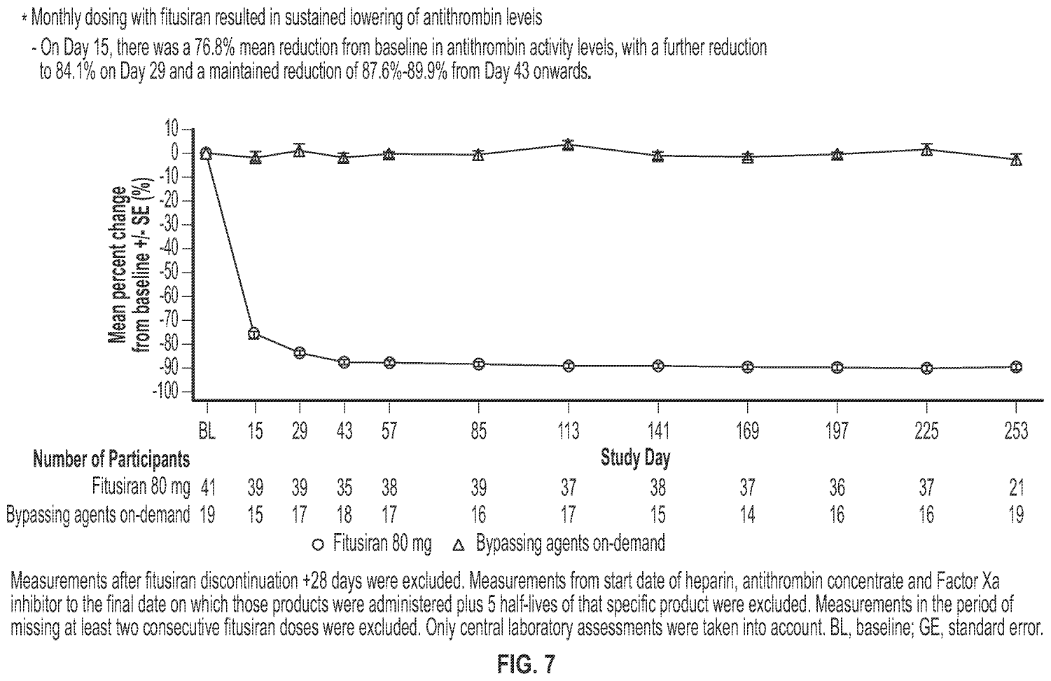

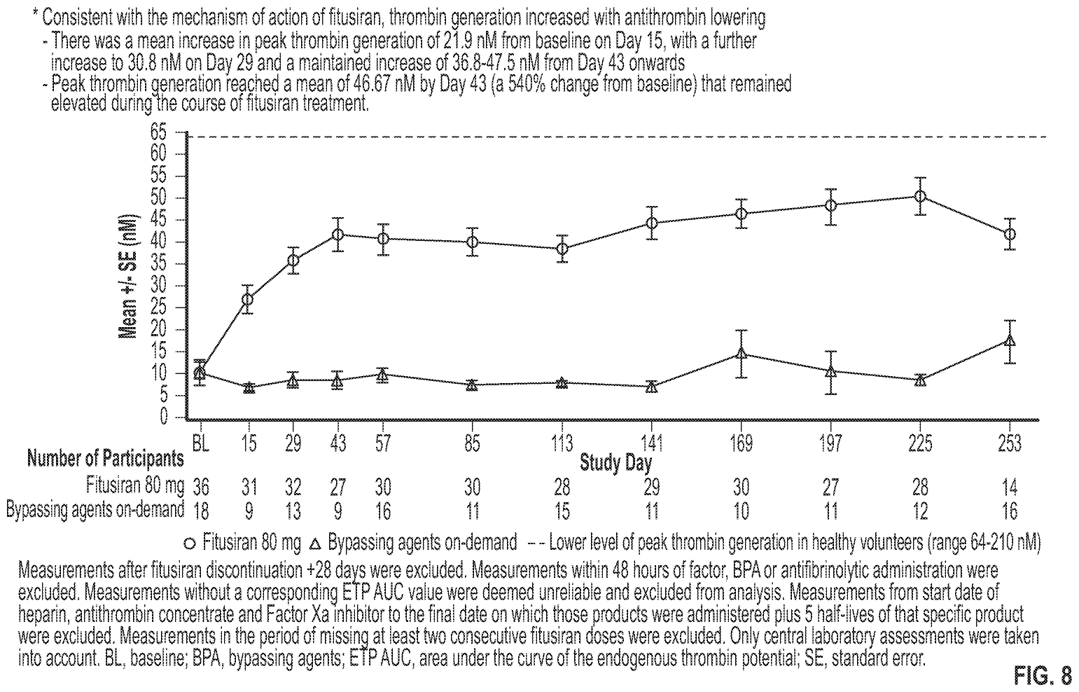

In patients with inhibitors, monthly dosing with fitusiran resulted in sustained lowering of antithrombin levels (FIG. 7). On day 29 there was a mean reduction of 84.1% from baseline in antithrombin activity levels and a maintained reduction of 87.6 to 89.9% from Day 43 onwards. Furthermore, thrombin generation increased with antithrombin lowering, with a mean increase in peak thrombin generation of 30.8 nM on day 29 and a maintained increase of 36.8 to 47.5 nM from day 43 onwards. Peak thrombin generation reached a mean of 46.67 nM by day 43 (FIG. 8), close to the lower level of peak thrombin generation of 64 nM previously observed in healthy volunteers, as illustrated by the dotted blue line on FIG. 8. Participants receiving fitusiran prophylaxis reached the target pharmacodynamic effect of antithrombin lowering and increased thrombin generation by day 29; these were sustained during the course of fitusiran treatment, demonstrating maintained and sustained hemostasis over the duration of the study. Fitusiran prophylaxis increased peak thrombin generation levels close to the lower levels previously observed in healthy volunteers.

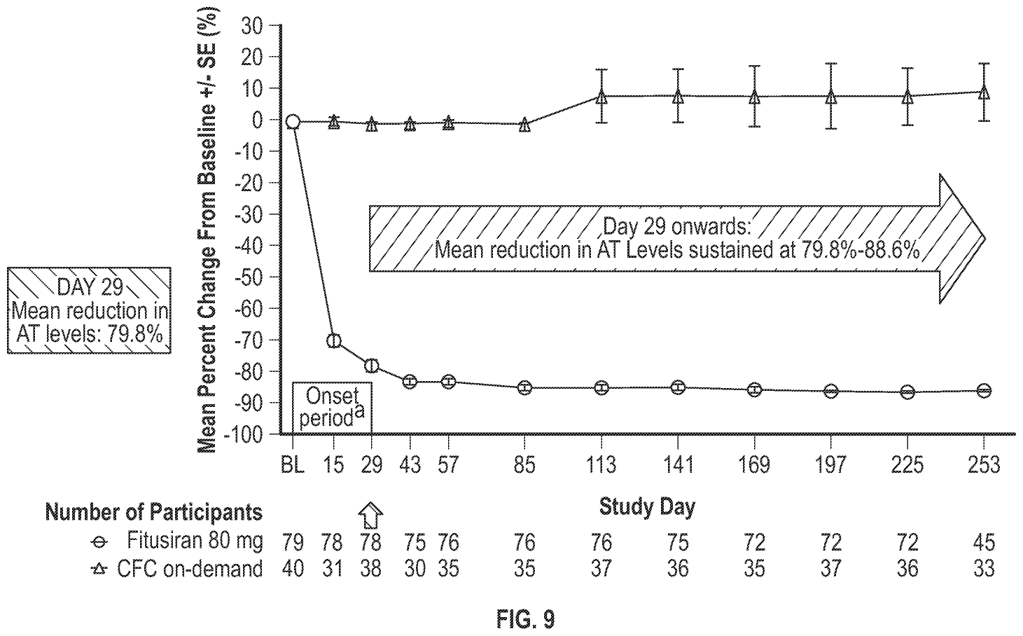

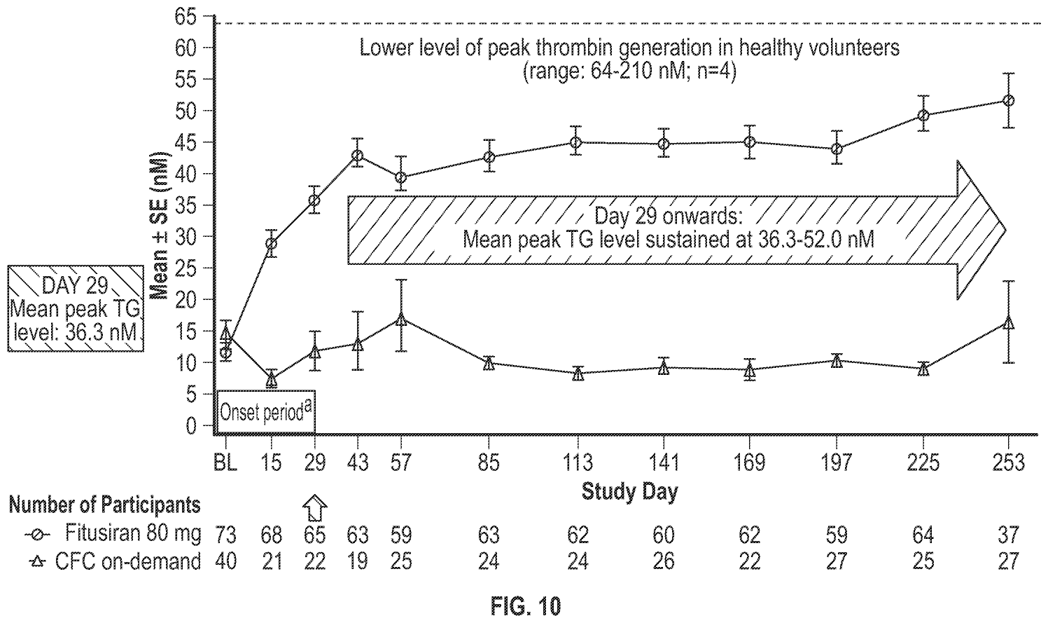

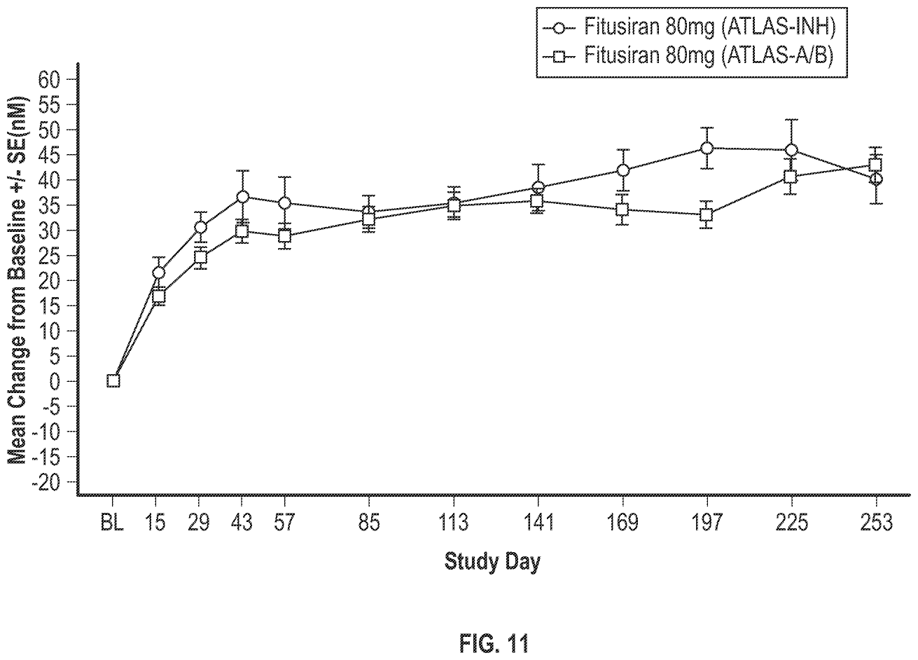

Similar results were observed for patients without inhibitors. On day 29 there was a mean reduction of 79.8% from baseline in antithrombin activity levels (FIG. 9). Fitusiran prophylaxis conferred a sustained reduction in antithrombin levels (FIG. 9) and a sustained increase in thrombin generation (FIG. 10). In patients without inhibitors, there was a mean increase in peak TG from baseline of 24.4 nM at day 29 (FIG. 11).

The mean change from baseline for peak thrombin generation in patients with (ATLAS-INH) or without (ATLAS-A/B) inhibitors is shown in FIG. 11. TG remained elevated during the entire study period. The present findings suggest fitusiran has the potential to rebalance hemostasis and provide sustained steady-state bleed protection in people with hemophilia A or B, with or without inhibitors.

### Safety and Tolerability

No treatment-emergent adverse events of thrombosis were reported in the Phase 3 study of patients without inhibitors (Table 10). Reported adverse events were generally consistent with previously identified risks of fitusiran.

TABLE 10
TEAEs in Patients Without inhibitors

| | | |
|---|---|---|
| | On-demand | Fitusiran |
| | Clotting Factor | 80 mg |
| | Concentrates | Prophylaxis |
| TEAE Category, n (%) | (n = 40) | (n = 79) |

| | | | | |
|---|---|---|---|---|
| Any TEAE | 18 | (45.0) | 62 | (78.5) |
| Any TESAE^{a} | 5 | (12.5) | 5 | (6.3) |

| | | | |
|---|---|---|---|
| Any TEAE leading to treatment | — | 2 | (2.5) |

| | | | | |
|---|---|---|---|---|
| discontinuation^{b} | | | | |
| Any TEAE leading to death | 0 | (0.0) | 0 | (0.0) |
| Any TEAESI | 1 | (2.5) | 15 | (19.0) |
| ALT increased | 0 | (0.0) | 12 | (15.2) |
| AST increased | 1 | (2.5) | 3 | (3.8) |
| Hepatic enzyme increased | 0 | (0.0) | 1 | (1.3) |
| Transaminases increased | 0 | (0.0) | 1 | (1.3) |

^{a}TESAEs in the fitusiran group included cholelithiasis (n = 2, 2.5%), cholecystitis (n = 1, 1.3%), lower respiratory tract infection (n = 1, 1.3%) and asthma (n = 1, 1.3%). TESAEs in the on-demand factor group included gastroenteritis, pneumonia, suicidal ideation, diplopia, osteoarthritis, epidural hemorrhage, humerus fracture, subdural hemorrhage and tibia fracture (all n = 1, 2.5%).
^{b}In the fitusiran group, 2 participants (2.5%) experienced TEAEs that resulted in fitusiran discontinuation (cholecystitis and ALT increased, in 1 participant each).

### Reduction in Bleed Rates

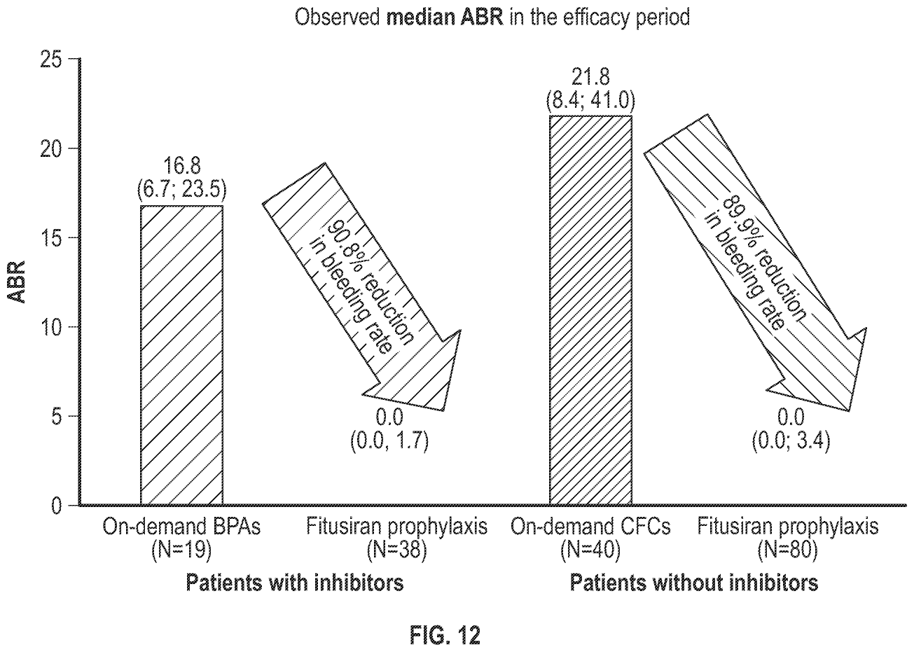

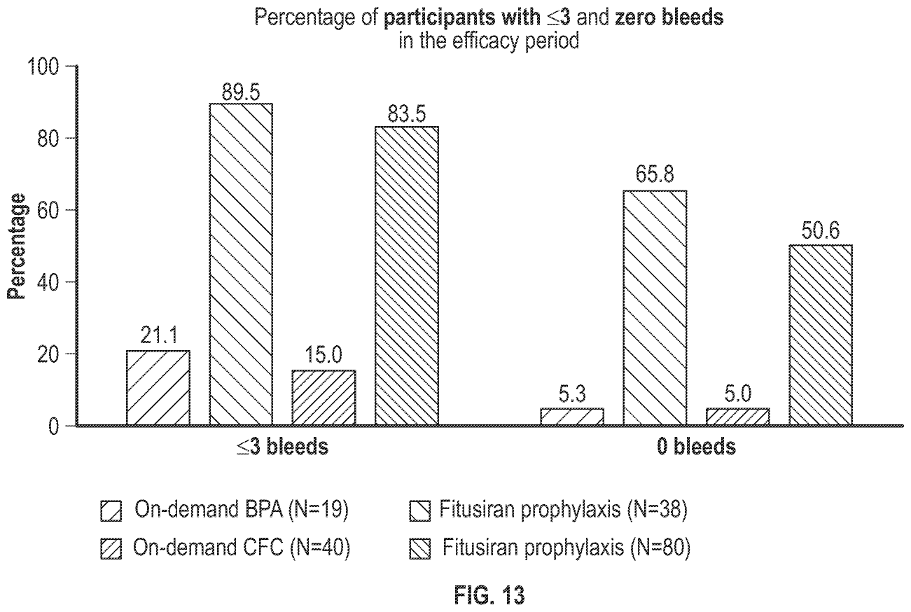

Fitusiran prophylaxis resulted in sustained protection from bleeds in people with hemophilia A or B, with or without inhibitors (FIGS. 12 and 13). The median ABR in each fitusiran treatment arm was 0.0.

### BPA and CFC Consumption

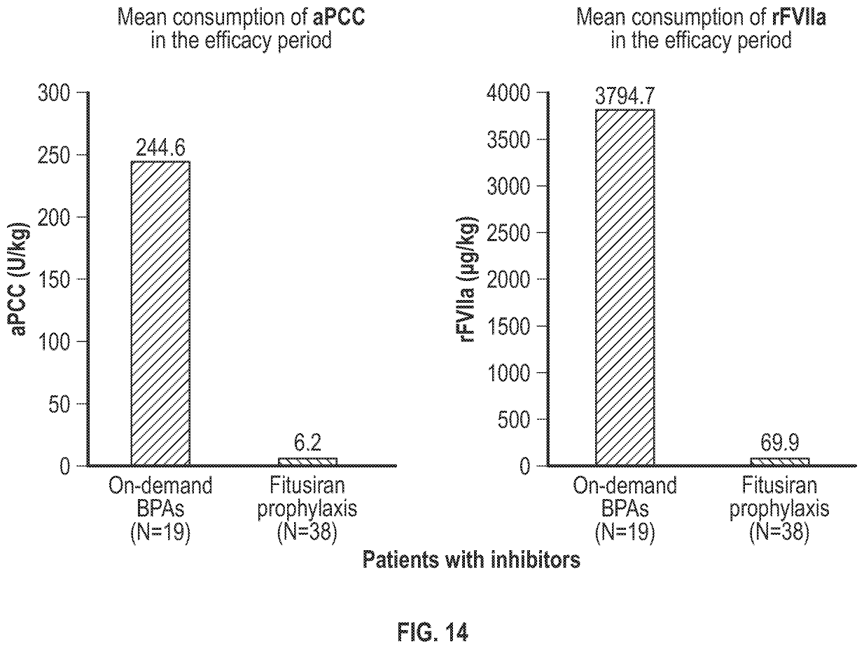

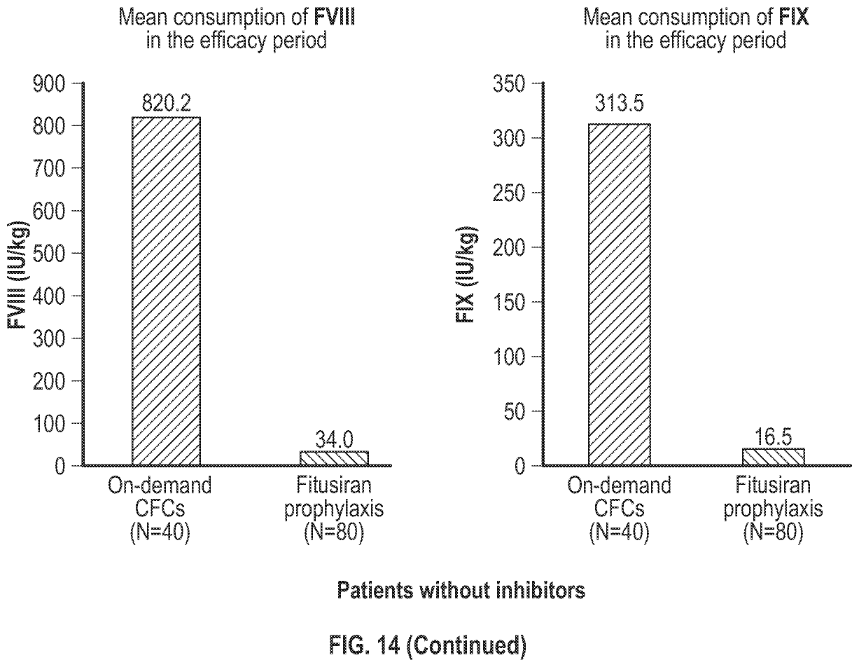

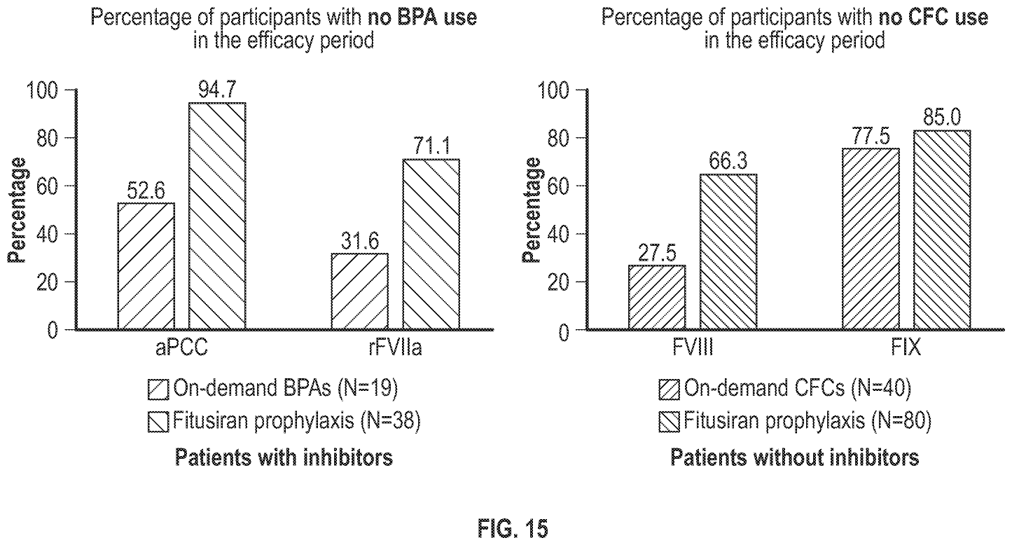

Overall mean consumption of aPCC and rFVIIa was 97.5% and 98.2% lower in the fitusiran arm compared to the on-demand BPA arm, and overall mean consumption of FVIII and FIX was lower in the fitusiran arm than the on-demand replacement factor arm (95.9% and 94.7%, respectively) (FIG. 14; Table 11).

TABLE 11
Summary of Annualized Weight-Adjusted BPA and Factor Concentrate Consumption During the Efficacy Period

| | Patients With Inhibitors | | | | Patients Without Inhibitors | | | |
|---|---|---|---|---|---|---|---|---|
| | | | Fitusiran 80 mg | | | | Fitusiran 80 mg | |
| | OD BPA | | prophylaxis | | OD CFC | | prophylaxis | |
| | (n = 19) | | (n = 38) | | (n = 40) | | (n = 80) | |
| | aPCC | rFVIIa | aPCC | rFVIIa | FVIII | FIX | FVIII | FIX |
| | (U/kg) | (ug/kg) | (U/kg) | (ug/kg) | (IU/kg) | (IU/kg) | (IU/kg) | (IU/kg) |
| Number of | 19 | 19 | 38 | 38 | 40 | 40 | 79 | 79 |
| participants* | | | | | | | | |
| Mean (SD) | 244.6 (381.3) | 3794.7 (9007.1) | 6.2 (32.4) | 69.9 (212.0) | 820.2 (1105.6) | 313.5 (916.8) | 34.0 (92.8) | 16.5 (50.2) |
| Median | 0.0 | 1533.0 | 0.0 | 0.0 | 398.5 | 0.0 | 0.0 | 0.0 |
| Q1; Q3 | 0.0; 448.1 | 0.0; 3186.8 | 0.0; 0.0 | 0.0; 69.3 | 0.0; 984.5 | 0.0; 0.0 | 0.0; 25.0 | 0.0; 0.0 |
| Min; Max | 0.0; 1294 | 0.0; 40102 | 0.0; 197 | 0.0; 1250 | 0.0; 3929 | 0.0; 5090 | 0.0; 634 | 0.0; 239 |

In the trial of patients with inhibitors, participants who received fitusiran required fewer mean injections (1.2 vs 3.7) and lower mean BPA doses per bleed compared to participants who received on-demand BPA (Table 12). In the trial of patients without inhibitors, participants in both arms required a mean 1.2 injections per bleed; participants who received fitusiran required lower mean replacement factor doses compared to participants who received on-demand replacement factor.

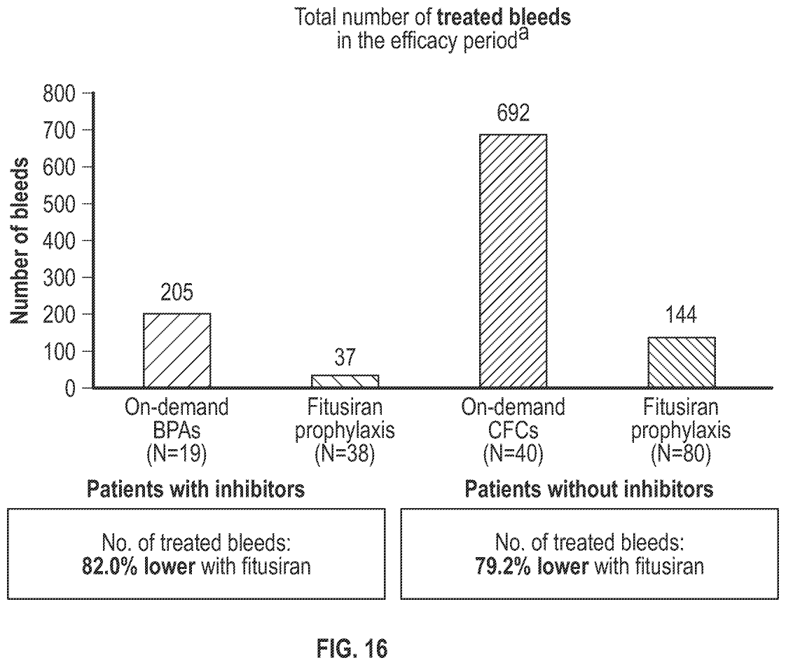

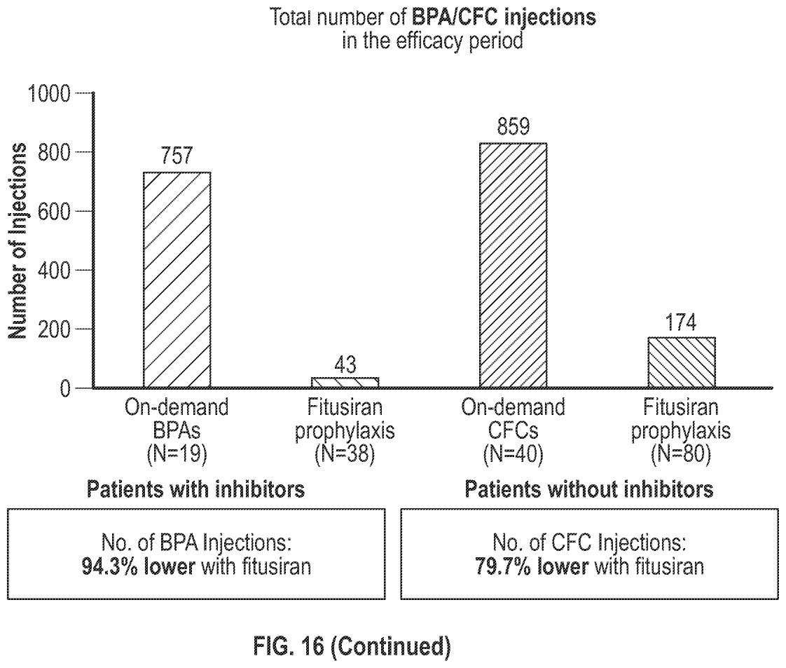

The proportion of participants with zero BPA/factor use was greater with fitusiran prophylaxis compared to on-demand BPA/factor treatment (FIG. 15), and the total number of treated bleeds and injections of BPA/factor was lower with fitusiran prophylaxis compared to on-demand BPA/factor (FIG. 16; Table 12). The total number of treated bleeds was lower in the fitusiran arm compared to the on-demand BPA and factor arms by 82.0% and 79.2%, respectively.

TABLE 12
Summary of BPA and Factor Injections for Treated Bleeds During the Efficacy Period

| | Patients With Inhibitors | | | | | |
|---|---|---|---|---|---|---|
| | OD BPA | | | Fitusiran 80 mg prophylaxis | | |
| | (n = 19) | | | (n = 38) | | |
| | aPCC | rFVIIa | | aPCC | rFVIIa | |
| | (U/kg) | (ug/kg) | Overall | (U/kg) | (ug/kg) | Overall |
| Number of | 9 (47.4) | 13 (68.4) | 18 (94.7) | 2 (5.3) | 11 (28.9) | 13 (34.2) |
| participants with | | | | | | |
| treated bleeds (%) | | | | | | |
| Total number of | 72 | 139 | 205 | 6 | 31 | 37 |
| treated bleeds | | | | | | |
| Mean number of | 1.3 (0.7) | 4.8 (7.2) | 3.7 (6.1) | 1.0 (0.0) | 1.2 (0.4) | 1.2 (0.4) |
| injections per bleed | | | | | | |
| (SD) | | | | | | |
| Mean total | 40.7 (23.1) | 449.8 (674.7) | — | 23.0 (9.9) | 57.7 (28.3) | — |
| weight-adjusted dose | | | | | | |
| per bleed (SD) | | | | | | |

| | Patients Without Inhibitors | | | | | |
|---|---|---|---|---|---|---|
| | OD CFC | | | Fitusiran 80 mg prophylaxis | | |
| | (n = 40) | | | (n = 80) | | |
| | FVIII | FIX | | FVIII | FIX | |
| | (IU/kg) | (IU/kg) | Overall | (IU/kg) | (IU/kg) | Overall |
| Number of | 29 (72.5) | 9 (22.5) | 38 (95.0) | 27 (33.8) | 12 (15.0) | 39 (48.8) |
| participants with | | | | | | |
| treated bleeds (%) | | | | | | |
| Total number of | 557 | 135 | 692 | 109 | 35 | 144 |
| treated bleeds | | | | | | |
| Mean number of | 1.2 (0.9) | 1.2 (0.6) | 1.2 (0.8) | 1.2 (0.6) | 1.1 (0.5) | 1.2 (0.6) |
| injections per bleed | | | | | | |
| (SD) | | | | | | |
| Mean total | 35.1 (25.2) | 51.8 (39.8) | — | 14.8 (10.9) | 21.5 (13.0) | — |
| weight-adjusted dose | | | | | | |
| per bleed (SD) | | | | | | |

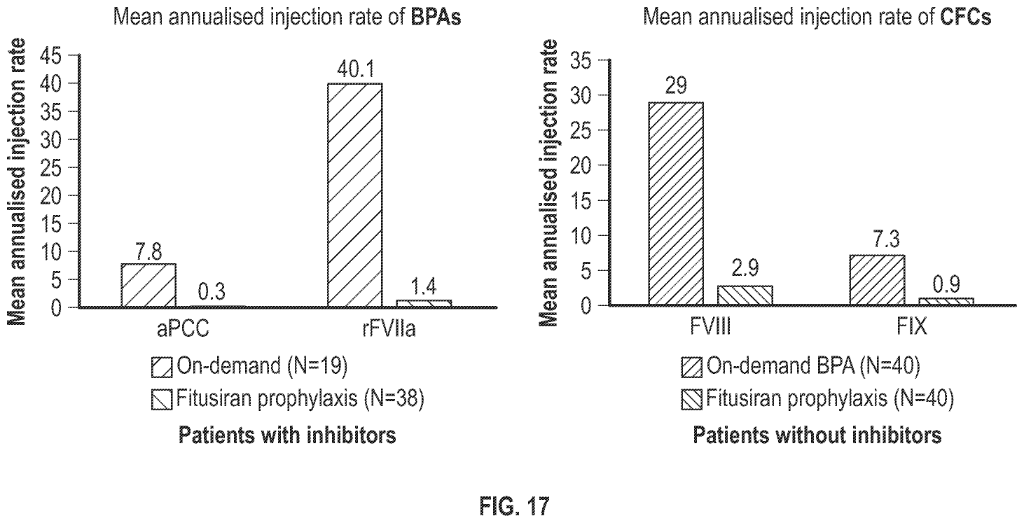

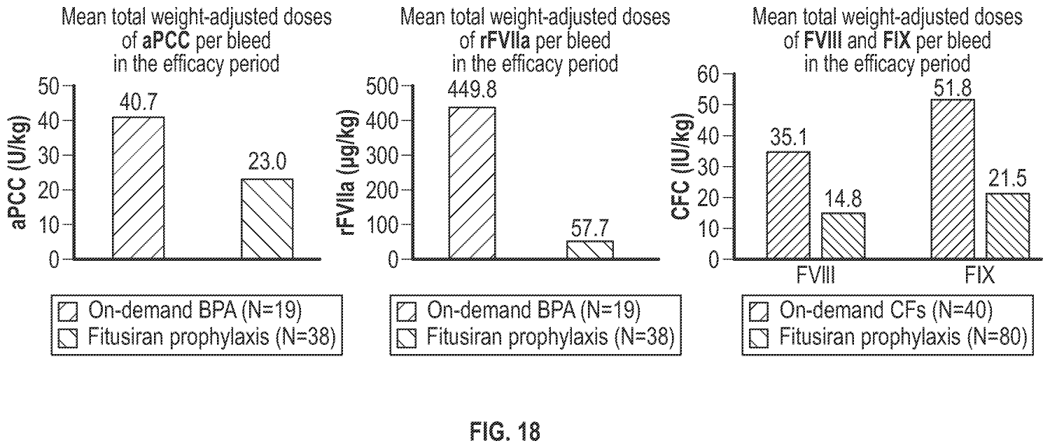

The mean annualized injection rate of BPAs and CFCs was lower with fitusiran prophylaxis treatment compared to on-demand BPA/CFC treatment (FIG. 17). Furthermore, the dose of BPAs/CFC required to treat bleeds was lower in patients treated prophylactically with fitusiran compared to on-demand BPA/CFC (FIG. 18; Table 12). In particular,
the aPCC dose was 43.5% lower with fitusiran,
the rFVIIa dose was 87.2% lower with fitusiran,
the FVIII dose was 57.8% lower with fitusiran, and
the FIX dose was 58.5% lower with fitusiran.

In sum, reduced consumption of BPAs/CFCs, as well as fewer injections and lower doses of BPAs and CFCs required to treat bleeds, was observed in patients undergoing fitusiran prophylaxis compared with on-demand/episodic BPA/CFC treatment.

### Example 4: Adolescent Subgroup Analysis

Overall, 33 adolescents (12-17 years) were enrolled (ten patients with inhibitors and 14 patients without inhibitors). Median ABR was 1.7 (patients with inhibitors) and 3.4 (patients without inhibitors) in the fitusiran arms, compared to 18.4 and 16.8, respectively, in the on-demand CFC and BPA arms. Fitusiran prophylaxis improved HRQoL with a mean change in transformed total score from baseline to month nine of −18.2 (patients with inhibitors) and −10.8 (patients without inhibitors) compared to −2.1 and 4.3, respectively, in the on-demand BPA and CFC treatment arms. The safety profile of fitusiran in adolescents was consistent with that observed in adult participants.

In sum, in adolescents with hemophilia A or B, with or without inhibitors, fitusiran prophylaxis demonstrated an improvement in bleeding phenotype. Decreases in Haemo-QoL scores support broad improvements in HRQoL with fitusiran prophylaxis compared with CFC and BPA treatments. The benefit/risk assessment was favorable.

### Example 5: Efficacy and Safety of Fitusiran Prophylaxis in People with Hemophilia A or B with Inhibitors (ATLAS-INH)

This multicenter, randomized, open-label Phase 3 study was conducted at 26 sites in 12 countries. Males aged ≥12 years with severe hemophilia A or B with inhibitors previously treated on-demand with bypassing agents (BPAs) were randomized 2:1 to receive once-monthly 80 mg subcutaneous fitusiran prophylaxis or to continue with BPAs on-demand for nine months. The primary endpoint was annualized bleeding rate (ABR) during the efficacy period. Safety and tolerability were assessed.

### Participants

Eligible participants were males aged ≥12 years with severe hemophilia A (defined by a central laboratory measurement or documented medical record evidence of FVIII <1% at screening) or hemophilia B (FIX ≤2%) and with inhibitory antibodies to FVIII or FIX (defined as Nijmegen-modified Bethesda assay ≥0.6 Bethesda units/mL at screening) who had experienced ≥6 bleeding episodes requiring on-demand BPA treatment within six months prior to screening.

Participants were excluded if they had antithrombin activity <60% at screening as determined by central laboratory measurement or a history of arterial or venous thromboembolism, atrial fibrillation, significant valvular disease, myocardial infarction, angina, transient ischemic attack, or stroke. Participants with a history of thrombosis associated with indwelling venous access were permitted to enroll. Written informed consent was obtained prior to the conduct of any trial-related procedures.

### Randomization and Masking

Eligible participants were randomized 2:1 to receive once-monthly 80 mg subcutaneously administered (0.8mL) fitusiran prophylaxis, with use of BPA on-demand for the treatment of breakthrough bleeds, or to continue with on-demand BPAs for the treatment of bleeding episodes. Participants were assigned to study groups by stratified permuted block randomization (block size of 3). On-demand use of BPAs was defined as the use of these agents, as needed, for episodic bleeding events (Table 13). Randomization was stratified by the number of bleeding episodes in the six months before screening (≤10 vs >10). The investigator or his/her delegate contacted the interactive response system after confirming that the participant fulfilled all inclusion criteria. The trial was open label, with both participants and investigators aware of treatment assignment.

TABLE 13
Breakthrough Bleed Management Dosing Guidelines

| Dose | aPCC | Recombinant Factor VIIa |
|---|---|---|
| Recommended | 30 U/kg | ≤45 μ/kg |
| single dose | | |
| Single dose | 50 U/kg | 45 μ/kg |
| should not exceed | | |
| Repeat dose | Mandatory to call the | Mandatory to call site |
| instructions | clinical study centre prior | prior to third dose |
| | to second dose | |
| | Consider evaluation and | |
| | treatment at the clinical | |
| | study centre | |
| | Should not repeat in less | Should not repeat in less |
| | than 24 hours | than 2 hours |
| | Should be seen at site | Should be seen at site |
| | within 48-72 hours if | within 48-72 hours if |
| | more than 2 doses are | more than 3 doses are |
| | required | required |

For situations requiring higher doses, more frequent administration, and multiple repeated doses, discussion with study Medical Monitor and Clinical Advisor is recommended, and antithrombin replacement should be considered.
Do not use antifibrinolytics in combination with factor or BPA.

### Procedures

The trial consisted of several periods: the onset period, from Days 1 to 28, during which fitusiran gradually reached the target pharmacodynamic effect of antithrombin lowering (Pasi et al., N Engl J Med (2017) 377(9):819-28); the efficacy period, from day 29 up to day 246, and the treatment period that consisted of both the onset and efficacy periods. The follow-up period lasted from one to six months, until antithrombin levels returned to approximately 60% following the final dose or until enrolment into an open-label extension study.

A treated bleeding episode was defined as any occurrence of hemorrhage that required administration of BPAs. The definitions of bleeding episode types were according to the recommendations of the International Society on Thrombosis and Hemostasis (Table 14)

TABLE 14
Definitions of Bleeding Episodes

| Variable | Definitions |
|---|---|
| Sponta- | A bleeding event that occurred for no apparent or known |
| neous | reason, particularly in the joints, muscles, and soft tissues. |
| Joint | A bleeding event characterized by an unusual sensation in the |
| | joint (“aura”) in combination with 1) increasing swelling |
| | or warmth over the skin over the joint, 2) increasing pain, or |
| | 3) progressive loss of range of motion or difficulty in using |
| | the limb as compared with baseline. |
| Muscle | A bleeding event characterized by pain, swelling and loss of |
| | movement over the affected muscle group. |
| Traumatic | A traumatic bleeding episode was caused by a known injury |
| | or trauma. |
| Target | A joint where 3 or more spontaneous bleeding episodes in a |
| joint | single joint within a consecutive 6-month period has occurred; |
| | where there have been ≤2 bleeding episodes in the joint |
| | within a consecutive 12-month period the joint is no longer |
| | considered a target joint. |

Health-related quality of life (HRQoL) was assessed at day 1 and at the end of the treatment period in adults aged 17 years or older using the validated Hemophilia Quality of Life Questionnaire for Adults (Haem-A-QoL) instrument (see, e.g., von Mackensen et al., Haemophilia (2017) 23(3):383-91).

Safety assessments consisted of treatment-emergent adverse events (TEAEs), including treatment-emergent serious adverse events (TESAEs) and treatment-emergent adverse events of special interest (TEAESIs); physical examinations, including vital signs and electrocardiograms; and laboratory tests, including markers of coagulation.

For pharmacodynamic assessments, antithrombin activity levels were measured from blood samples collected within four hours before dosing and monitored at monthly intervals from day 1 until returning to an approximate activity level of 60% after the final fitusiran dose (unless participants opted to continue fitusiran in an open-label extension study). Thrombin generation was also assessed at monthly intervals. Antidrug antibodies (ADA) to fitusiran were measured from serum blood samples collected within 4 hours of treatment administration on day 1, months 1 and 3, and at end of treatment using a validated enzyme linked immunosorbent assay method.

### Outcomes

The primary efficacy endpoint was ABR in the efficacy period. ABR was defined as the number of qualifying bleeding episodes divided by the total number of days in the efficacy period multiplied by 365.25. Pre-specified subgroup analyses of the primary endpoint were performed according to hemophilia type (Type A vs B), number of bleeding episodes during the 6 months prior to study (≤10 vs >10) and age group (<18, 18-64, ≥65 years) in the efficacy period. Secondary endpoints included ABR in the treatment period, annualized spontaneous bleeding rate (AsBR) in the efficacy period, and annualized joint bleeding rate (AjBR) in the efficacy period. Change from baseline in Haem-A-QoL physical health domain and total score in the treatment period were also assessed as a secondary endpoint.

Safety and tolerability endpoints included incidence, severity, seriousness, and relatedness of adverse events. TEAESIs were pre-defined as alanine aminotransferase (ALT) or aspartate aminotransferase elevations (AST) >3×upper limit of normal (ULN), suspected or confirmed thrombosis, severe or serious injection site reactions, and systemic injection-associated reactions.

Pre-specified exploratory endpoints included number of target joint bleeding episodes in the efficacy period, pharmacokinetic and pharmacodynamic effects of fitusiran (including antithrombin activity and thrombin generation), and incidence and titer of ADA in the fitusiran prophylaxis group. There was no allowance for multiplicity for the exploratory outcomes.

### Determination of Sample Size

Using a negative binomial regression model with a two-sided type I error rate of 0.05, an estimated sample size of 14 participants in the BPA on-demand group (assuming mean [SD] ABR of 18 [14]) and 28 participants in the fitusiran prophylaxis group (assuming mean [SD] ABR of 4 [6]) was projected to provide >90% power for detecting treatment difference in the primary endpoint. The planned sample size was 54 randomized participants assuming a 20% drop-out rate.

### Statistical Analysis

All statistical analyses were performed using SAS statistical software Version 9.4. The primary analyses were performed on the intent-to-treat analysis set (all randomized participants). Before any Chinese participants could be randomized to receive 80 mg subcutaneously administered fitusiran prophylaxis, the first three participants were enrolled into a China-specific non-randomized fitusiran group to receive subcutaneously administered fitusiran prophylaxis every four weeks, with an initial starting dose of 50 mg escalating to 80 mg following an observation period, as planned in the protocol to comply with the regulatory requirement of the FDA board in China. These participants were not included in the intent- to-treat population but were included in the safety set.

The number of bleeding episodes in the efficacy period was analyzed using a negative binomial model with fixed effects of treatment group and the number of bleeding episodes in the six months prior to study entry (≤10 vs >10). The logarithm number of days that each participant spent in the efficacy period matching the bleeding episode data being analyzed was included as an offset variable to account for unequal follow-up time due to early withdrawal, surgery, etc. The estimated mean ABR in both treatment groups along with their 95% confidence intervals (CIs) were presented from this model. Summary statistics for ABR, including median and IQR, were also calculated for each group. Sensitivity analyses were performed for the efficacy outcomes.

AsBR and AjBR were analyzed using the same methodology as the primary analysis. Least square (LS) mean change from baseline in physical health score and total score of Haem-A-QoL were analyzed using an analysis of covariance model with fixed effects of treatment group, baseline Haem-A-QoL physical health score and total score, and number of bleeding episodes six months before enrolment (≤10 vs >10). To control for the familywise error rate in the testing of primary and secondary endpoints, a hierarchical testing approach was used.

Safety, pharmacokinetic, pharmacodynamic, and immunogenicity results were summarized descriptively. In the safety population, all participants received at least one dose of fitusiran or were randomized to the on-demand group; all by-treatment analyses based on the safety analysis set were according to the actual treatment received.

The trial was registered at www.clinicaltrials.gov under identifier NCT03417102.

### Results

A total of 85 participants were screened, 25 of whom failed screening and three of whom were not randomized, and 57 participants were randomized to the BPA on-demand group (n=19) and the fitusiran prophylaxis group (n=38). All randomized participants completed the study. Baseline demographics and clinical characteristics were similar between both groups in the safety set (Table 15); most participants had hemophilia A (n=48, 80%) and multiple target joints present at baseline (67% of participants had ≥1 target joint). The median duration of fitusiran exposure was 252.0 days (minimum 203 days; maximum 271 days).

TABLE 15
Demographics and Baseline Characteristics - ITT Safety set

| | BPA | | Fitusiran 80 mg | | | |
|---|---|---|---|---|---|---|
| | on-demand | | prophylaxis | | All | |
| Characteristic | (N = 19) | | (N = 41)^{†} | | (N = 60) | |
| Age, years, median (IQR) | 28.0 | (23.0; 36.0) | 26.0 | (17.0; 32.0) | 27.0 | (19.5; 33.5) |
| Weight, kg, mean (SD) | 70.2 | (17.6) | 66.1 | (18.5) | 67.4 | (18.2) |
| BMI, kg/m², mean (SD) | 25.2 | (5.8) | 23.5 | (5.5) | 24.1 | (5.6) |
| Race, n (%) | | | | | | |
| White | 6 | (31.6) | 10 | (24.4) | 16 | (26.7) |
| Asian | 13 | (68.4) | 29 | (70.7) | 42 | (70.0) |
| Other | 0 | | 1 | (2.4) | 1 | (1.7) |
| Multiple | 0 | | 1 | (2.4) | 1 | (1.7) |
| Region, n (%) | | | | | | |
| North America | 0 | | 6 | (14.6) | 6 | (10.0) |
| Europe | 4 | (21.1) | 7 | (17.1) | 11 | (18.3) |
| Asia | 13 | (68.4) | 28 | (68.3) | 41 | (68.3) |
| Other | 2 | (10.5) | 0 | | 2 | (3.3) |
| Number of bleeding episodes in the | 10.0 | (7.0; 15.0) | 10.0 | (7.0; 16.0) | 10.0 | (7.0; 16.0) |
| last 6 months prior to screening, | | | | | | |
| median (IQR) | | | | | | |
| Haemophilia type, n (%) | | | | | | |
| Haemophilia A | 16 | (84.2) | 32 | (78.0) | 48 | (80.0) |
| Haemophilia B | 3 | (15.8) | 9 | (22.0) | 12 | (20.0) |
| Inhibitor titre (highest historical | | | | | | |
| inhibitor titre result*), n (%) | | | | | | |
| <5 BU/mL | 1 | (5.3) | 6 | (14.6) | 7 | (11.7) |
| Haemophilia A | 1 | (5.3) | 3 | (7.3) | 4 | (6.7) |
| Haemophilia B | 0 | | 3 | (7.3) | 3 | (5.0) |
| ≥5 BU/mL | 18 | (94.7) | 35 | (85.4) | 53 | (88.3) |
| Haemophilia A | 15 | (78.9) | 29 | (70.7) | 44 | (73.3) |
| Haemophilia B | 3 | (15.8) | 6 | (14.6) | 9 | (15.0) |
| Number of target joints, median | 1.0 | (0.0; 3.0) | 1.0 | (0.0; 2.0) | 1.0 | (0.0; 2.0) |
| (IQR) | | | | | | |

*Bethesda assay
^{†}Three participants in China were treated but not randomised; participants were enrolled into a China-specific non-randomised fitusiran group to receive subcutaneous fitusiran prophylaxis every 4 weeks, with initial starting dose of 50 mg.
One participant had a reported medical history of thrombosis associated with indwelling venous access. This participant did not experience a TEAESI of ‘suspected or confirmed thrombosis’ while enrolled in the study.
BMI, body mass index;
BPA, bypassing agent;
BU, Bethesda unit;
IQR, interquartile range;
ITT, intent-to-treat;
rFVIIa, recombinant activated factor VII;
SD, standard deviation

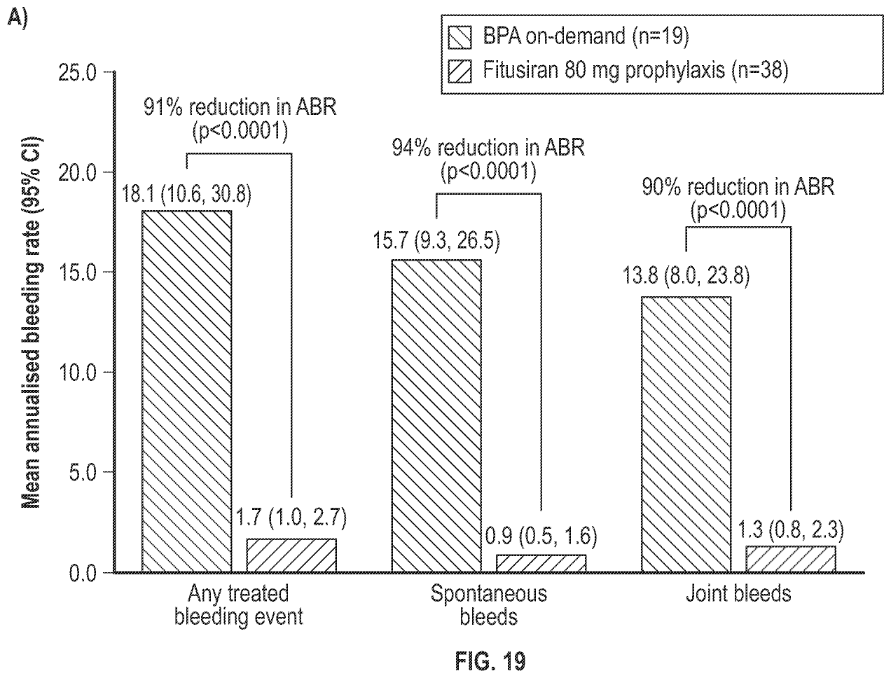

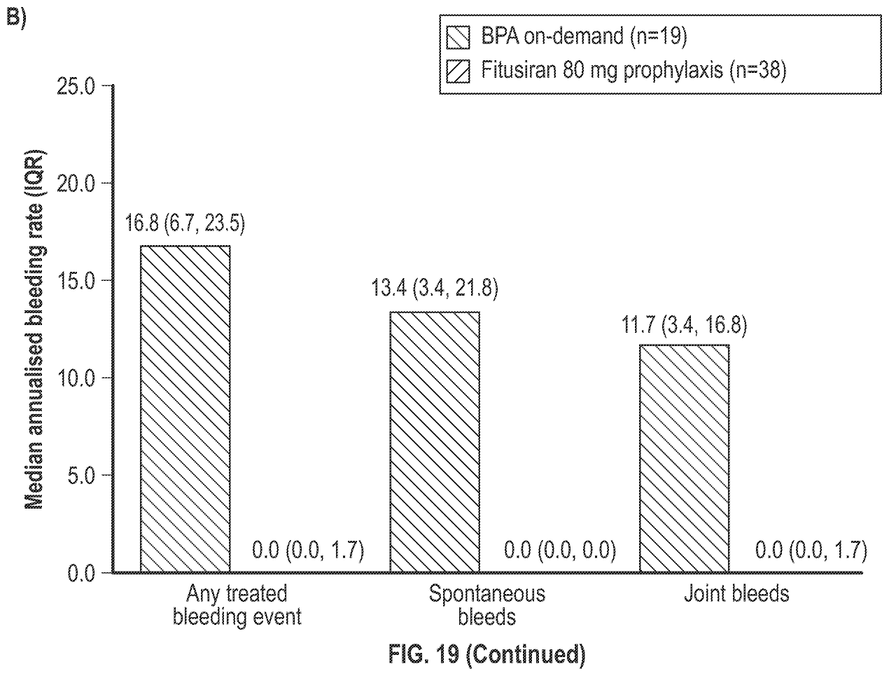

Mean ABR estimated by the negative binomial model in the efficacy period was statistically significantly lower in the fitusiran prophylaxis group (1.7 [95% CI: 1.0, 2.7]) compared with the BPA on-demand group (18.1 [95% CI: 10.6, 30.8]; Table 16, FIG. 19), corresponding to a statistically and clinically significant reduction in bleeding rate of 90.8% (95% CI: 80.8, 95.6%) in favor of fitusiran prophylaxis (p<0.0001). Median ABRs were consistent with these findings: 0.0 (IQR: 0.0, 1.7) in the fitusiran prophylaxis group and 16.8 (IQR: 6.7, 23.5) in the BPA on-demand group (Table 16, FIG. 19). In the efficacy period, 25 participants (66%) had zero treated bleeds in the fitusiran prophylaxis group versus one (5%) in the BPA on-demand group. In the fitusiran arm, 13 participants experienced a treated bleed, two of whom received aPCC and 11 received rFVIIa. In the BPA on-demand group, 18 participants experienced a treated bleed, nine of whom received aPCC and 13 of whom received rFVIIa (n=4 received both). Annualized bleeding rate in the treatment period demonstrated similar results to the primary endpoint. When analyzed by hemophilia subtypes, median ABRs in the efficacy period were lower in the fitusiran prophylaxis group compared with BPA on demand group in both patients with hemophilia A (0.0 [IQR: 0.0, 0.0] versus 15.9 [IQR: 6.7, 23.5], respectively) and hemophilia B (1.7 [IQR: 0.0, 5.0] versus 18.4 [IQR: 5.1, 23.5], respectively) (Table 17).

TABLE 16
Primary and Secondary Efficacy Outcomes - ITT set

| | | | |
|---|---|---|---|
| | | Fitusiran 80 mg | |
| | BPA on-demand | prophylaxis | |
| Outcome | (N = 19) | (N = 38) | P-value |

Primary efficacy outcome

| | | | | | |
|---|---|---|---|---|---|
| All treated bleeds (efficacy | | | | | |
| period) | | | | | |
| Mean ABR estimated by negative | 18.1 | (10.6; 30.8) | 1.7 | (1.0; 2.7) | p < 0.0001 |
| binomial model (95% CI)* | | | | | |
| Observed median ABR (IQR) | 16.8 | (6.7; 23.5) | 0.0 | (0.0; 1.7) | |
| Participants with zero bleeds, n (%) | 1 | (5.3) | 25 | (65.8) | |

Secondary efficacy outcomes

| | | | | | |
|---|---|---|---|---|---|
| All treated bleeds (treatment | | | | | |
| period) | | | | | |
| Mean ABR estimated by negative | 18.8 | (11.5; 30.7) | 2.0 | (1.3; 3.1) | p < 0.0001 |
| binomial model (95% CI)* | | | | | |
| Observed median ABR (IQR) | 16.3 | (5.9; 23.8) | 0.0 | (0.0; 1.6) | |
| Participants with zero bleeds, n (%) | 1 | (5.3) | 20 | (52.6) | |
| Treated spontaneous bleeds | | | | | |
| (efficacy period) | | | | | |
| Mean AsBR estimated by negative | 15.7 | (9.3, 26.5) | 0.9 | (0.5, 1.6) | p < 0.0001 |
| binomial model (95% CI)* | | | | | |
| Observed median AsBR (IQR) | 13.4 | (3.4; 21.8) | 0.0 | (0.0; 0.0) | |
| Participants with zero bleeds, n (%) | 2 | (10.5) | 29 | (76.3) | |
| Treated joint bleeds (efficacy | | | | | |
| period) | | | | | |
| Mean AjBR estimated by negative | 13.8 | (8.0; 23.8) | 1.3 | (0.8; 2.3) | p < 0.0001 |
| binomial model (95% CI)* | | | | | |
| Observed median AjBR (IQR) | 11.7 | (3.4; 16.8) | 0.0 | (0.0; 1.7) | |
| Participants with zero bleeds, n (%) | 1 | (5.3) | 27 | (71.1) | |
| Change in Haem-A-QoL | | | | | |
| transformed scores in the | | | | | |
| treatment period | | | | | |
| Physical health score | −1.9 | (−10.3; 6.4) | −30.7 | (−36.9; −24.4) | p < 0.0001 |
| LS Mean (95% CI) | | | | | |
| Total score | −0.42 | (−5.7; 4.8) | −15.3 | (−19.3; −11.2) | p < 0.0001 |
| LS mean (95% CI) | | | | | |

TABLE 17
Bleeding Events in the Efficacy Period Based on Hemophilia
Subtype (Pre-Specified Subgroup Analysis)

| | | Fitusiran 80 mg | |
|---|---|---|---|
| Event | BPA on-demand | prophylaxis | P-value |
| Participants with hemophilia A, n | 16 | 29 | |

Any treated bleeding event

| | | | | | |
|---|---|---|---|---|---|
| Median ABR (IQR) | 15.9 | (6.7; 23.5) | 0.0 | (0.0; 0.0) | |
| Mean ABR estimated by negative binomial | 18.3 | (9.7; 34.5) | 1.3 | (0.7; 2.5) | p < 0.0001 |
| model (95% CI)* | | | | | |
| Participants with zero treated bleeds, n (%) | 1 | (6.3) | 22 | (75.9) | |

Treated spontaneous bleeds

| | | | | | |
|---|---|---|---|---|---|
| Median ABR (IQR) | 12.6 | (5.0; 21.8) | 0.0 | (0.0; 0.0) | |
| Mean ABR estimated by negative binomial | 16.2 | (8.7; 29.9) | 0.80 | (0.4; 1.6) | p < 0.0001 |
| model (95% CI)* | | | | | |

Treated joint bleeds

| | | | | | |
|---|---|---|---|---|---|
| Median ABR (IQR) | 12.6 | (3.4; 17.6) | 0.0 | (0.0; 0.0) | |
| Mean ABR estimated by negative binomial | 14.4 | (7.6; 27.4) | 1.2 | (0.6; 2.3) | p < 0.0001 |
| model (95% CI)* | | | | | |

| | | |
|---|---|---|
| Participants with hemophilia B, n | 3 | 9 |

Any treated bleeding event

| Median ABR (IQR) | 18.4 | (5.1; 23.5) | 1.7 | (0.0; 5.0) | |
|---|---|---|---|---|---|
| Mean ABR estimated by negative binomial | N/A | | N/A | | N/A |
| model (95% CI)* | | | | | |

| | | | |
|---|---|---|---|
| Participants with zero treated bleeds, n (%) | 0 | 3 | (33.3) |

Treated spontaneous bleeds

| Median ABR (IQR) | 13.4 | (1.7; 18.4) | 0.0 | (0.0; 1.7) | |
|---|---|---|---|---|---|
| Mean ABR estimated by negative binomial | N/A | | N/A | | N/A |
| model (95% CI)* | | | | | |

Treated joint bleeds

| | | | | | |
|---|---|---|---|---|---|
| Median ABR (IQR) | 8.4 | (5.1; 16.8) | 0.0 | (0.0; 5.0) | |
| Mean ABR estimated by negative binomial | 8.8 | (4.9; 16.1) | 1.8 | (0.9; 3.4) | p = 0.0002 |
| model (95% CI)* | | | | | |

*Negative binomial regression model includes treatment group, randomisation strata of number of bleeds in the 6 months prior to study (≤10, >10) as fixed effects, and the logarithm of the duration that each participant spent in the efficacy period matching the bleeding episode being analysed as an offset variable. Results were provided for subgroups that had at least three participants in each treatment group and the model converges.
ABR, annualised bleeding rate;
BPA, bypassing agent;
CI, confidence interval;
IQR, interquartile range

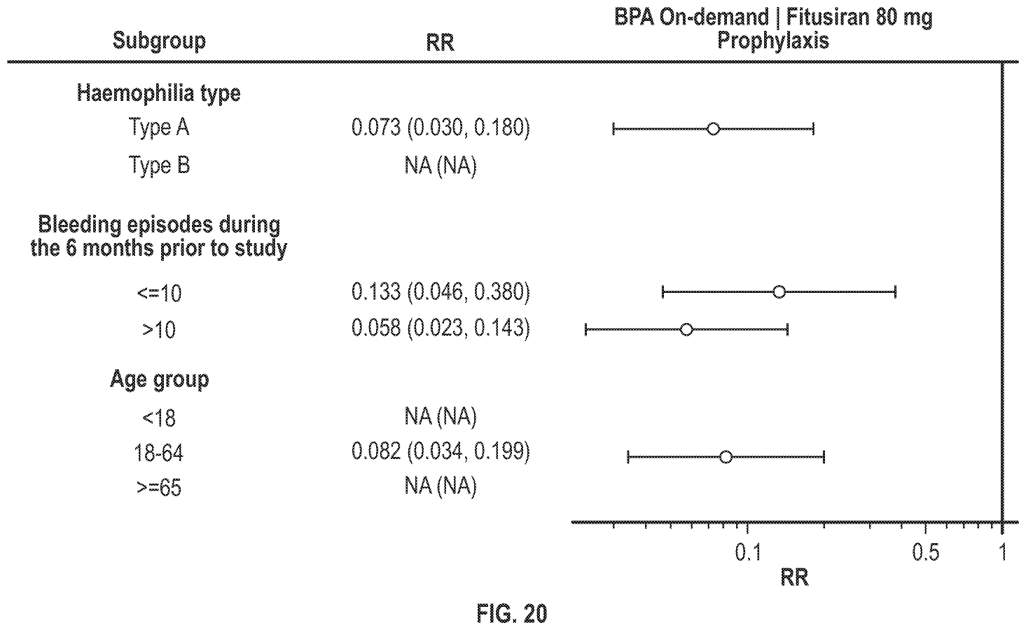

Subgroup analyses confirmed the primary endpoint. In all subgroups, estimated ABR results favored fitusiran prophylaxis (FIG. 20, Table 17). The results of the sensitivity analyses supported the primary efficacy outcomes and are reported in Table 18.

TABLE 18
Sensitivity Analyses of Bleeding Events in the Efficacy
Period - Pre-Specified Sensitivity Analyses

| | | | Fitusiran 80 mg | | |
|---|---|---|---|---|---|
| Event | BPA on-demand | | prophylaxis | | P-value |
| IIT analysis set sensitivity analysis, n | 19 | | 38 | | |
| Median ABR (IQR) | 16.8 | (6.7; 23.5) | 0.0 | (0.0; 1.7) | |
| Mean ABR estimated by negative binomial | 18.0 | (10.6; 30.8) | 1.8 | (1.1; 3.0) | p < 0.0001 |
| model (95% CI)* | | | | | |
| Participants with zero treated bleeds, n (%) | 1 | (5.3) | 25 | (65.8) | |
| Per-protocol set, n | 19 | | 37 | | |
| Median ABR (IQR) | 16.8 | (6,7; 23.5) | 0.0 | (0.0; 1.7) | |
| Mean ABR estimated by negative binomial | 18.1 | (10.5; 31.1) | 1.7 | (1.0; 2.8) | p < 0.0001 |
| model (95% CI)* | | | | | |
| Participants with zero treated bleeds, n (%) | 1 | (5.3) | 25 | (67.6) | |
| COVID-19 unaffected set, n | 14 | | 25 | | |
| Median ABR (IQR) | 17.6 | (5.0; 23.5) | 0.0 | (0.0; 1.7) | |
| Mean ABR estimated by negative binomial | 18.9 | (9.3; 38.3) | 1.7 | (0.9; 3.5) | p < 0.0001 |
| model (95% CI)* | | | | | |
| Participants with zero treated bleeds, n (%) | 1 | (7.1) | 17 | (68.0) | |
| Zero-inflated negative binomial model, n | 19 | | 38 | | |
| Mean ABR estimated by negative binomial | 18.1 | (12.2; 26.8) | 3.7 | (2.0; 7.1) | p < 0.0001 |
| model (95% CI)* | | | | | |

*Negative binomial regression model includes treatment group, randomisation strata of number of bleeds in the 6 months prior to study (≤10, >10) as fixed effects, and the logarithm of the duration that each participant spent in the efficacy period matching the bleeding episode being analysed as an offset variable.
ABR, annualized bleeding rate;
BPA, bypassing agent;
CI, confidence interval;
IQR, interquartile range

In the efficacy period, the median AsBRs were 0.0 (IQR: 0.0, 0.0) and 13.4 (IQR: 3.4, 21.8) in the fitusiran prophylaxis and BPA on-demand groups, respectively. A greater proportion of participants in the fitusiran prophylaxis group had zero spontaneous bleeds (76%) versus the BPA on-demand group (11%). Median AjBRs were 0.0 (IQR: 0.0, 1.7) and 11.7 (IQR: 3.4, 16.8) in the fitusiran prophylaxis and BPA on-demand groups, respectively. In the fitusiran prophylaxis group, 11 (29%) participants had ≥1 joint bleed, compared with 18 (95%) participants in the BPA on-demand group; two (5%) and 13 (68%) participants, respectively, had >3 joint bleeds. In participants with ≥1 target joint identified at baseline, 4/26 (15%) and 12/12 (100%) participants in the fitusiran prophylaxis and BPA on-demand groups, respectively, had a treated spontaneous target joint bleeding event during the efficacy period. Most participants (85%) in the fitusiran prophylaxis group had zero target joint bleeding events compared with no participants with zero target joints bleeds (0%) in the BPA on-demand group (Table 19).

TABLE 19
Number of Target Joint Bleeding Episodes
in the Efficacy Period - ITT Set

| | BPA | | Fitusiran | | |
|---|---|---|---|---|---|
| | on-demand | | prophylaxis | | |
| | (N = 19) | | (N = 38) | | P-value |
| Mean ABR estimated by | 2.5 | (1.7; 3.6) | 0.2 | (0.1; 0.4) | p < 0.0001 |
| negative binomial | | | | | |
| model (95% CI)* | | | | | |
| Observed median ABR | 2.8 | (1.0; 3.9) | 0.0 | (0.0; 0.0) | |
| (IQR) | | | | | |
| Participants with zero | 0 | (0) | 22 | (84.6) | |
| bleeds, n (%) | | | | | |

*Negative binomial regression model includes treatment group, randomization strata of number of bleeds inthe 6 months prior to study (≤10, >10) as fixed effects, and the logarithm of the duration that each participantspent in the efficacy period matching the bleeding episode being analyzed as an offset variable
ABR, annualized bleeding rate; BPA, bypassing agents; CI, confidence intervals; IQR, interquartile range; SD, standard deviation.

The percentage of participants with major protocol deviations identified during the study was similar between treatment arms, 68.4% (n=13) in the BPA on-demand group, and 76.3% (n=29) in the fitusiran prophylaxis arm.

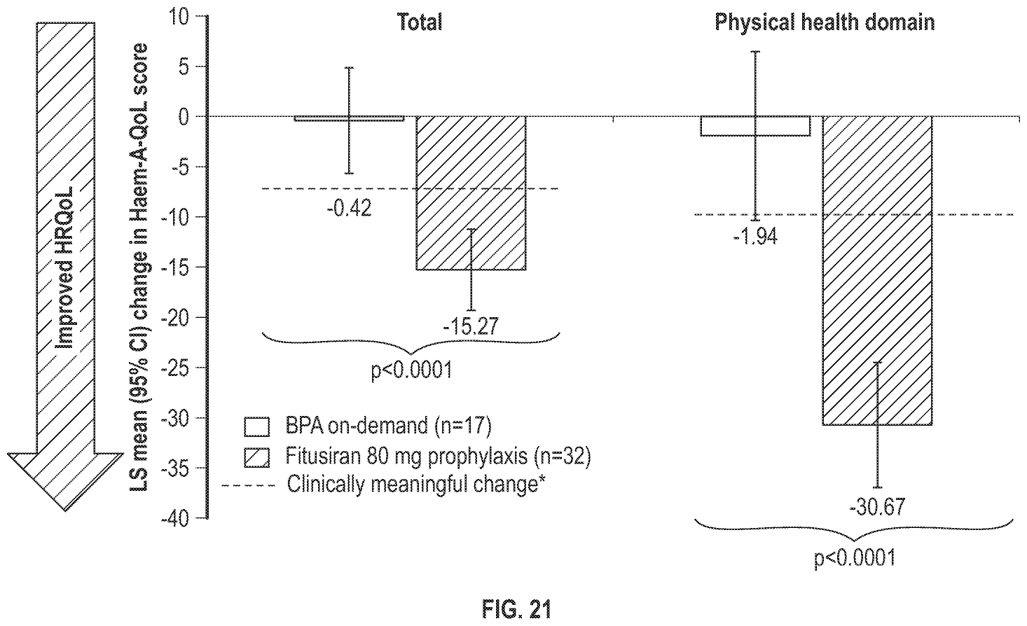

There were statistically significant reductions (improvements) in LS mean (95% CI) Haem-A-QoL transformed physical health domain score relative to baseline in the fitusiran prophylaxis group (−30.7 [−36.9, −24.4]) compared with the BPA on-demand group (−1.9 [−10.3, 6.4]), with a LS mean difference of 31 28.7 (−39.1, −18.4) in favor of fitusiran prophylaxis (p<0.0001) (FIG. 21). A statistically significant reduction was also observed in the Haem-A-QoL transformed total score in the fitusiran prophylaxis group (−15.3 [−19.3, −11.2]) compared with the BPA on-demand group (−0.4 [−5.7, 4.8]), with a LS mean difference of −14.9 (−21.4, −8.3) in favour of fitusiran prophylaxis (p<0.0001) (FIG. 21).

Overall, 38 (93%) participants in the fitusiran prophylaxis group and 11 (58%) participants in the BPA on-demand group experienced at least one TEAE; the most common TEAEs (reported in >5% of participants in the fitusiran prophylaxis group) are summarized in Table 20. TESAEs were reported in seven participants (17%) in the fitusiran prophylaxis group and five participants (26%) in the BPA on-demand group. In the fitusiran prophylaxis group, one participant (2%) experienced two TESAEs that were assessed by the investigator as possibly related to fitusiran and that resulted in study drug discontinuation (spinal vascular disorder and thrombosis [suspected spinal vessel thrombosis]). There were no TEAEs that resulted in study withdrawal and no TEAEs leading to death.

TABLE 20
Summary of Treatment-Emergent Adverse Events - Safety Set

| | | | Fitusiran | |
|---|---|---|---|---|
| | BPA | | 80 mg | |
| | on-demand | | prophylaxis | |
| n (%) | (N = 19) | | (N = 41)* | |
| Participants with any TEAE | 11 | (57.9) | 38 | (92.7) |
| Most frequent (>5%) TEAEs in fitusiran | | | | |
| group | | | | |
| ALT increased | 0 | | 13 | (31.7) |
| AST increased | 0 | | 8 | (19.5) |
| Upper respiratory tract infection | 1 | (5.3) | 6 | (14.6) |
| Headache | 1 | (5.3) | 6 | (14.6) |
| Abdominal pain upper | 0 | | 6 | (14.6) |
| Gamma-glutamyl transferase increased | 0 | | 6 | (14.6) |
| Arthralgia | 0 | | 5 | (12.2) |
| Blood alkaline phosphatase increased | 0 | | 5 | (12.2) |
| Transaminases increased | 0 | | 5 | (12.2) |
| Influenza | 0 | | 3 | (7.3) |
| Nasopharyngitis | 0 | | 3 | (7.3) |
| Pyrexia | 0 | | 3 | (7.3) |
| Fibrin D dimer increased | 0 | | 3 | (7.3) |
| Prothrombin fragment 1.2 increased | 0 | | 3 | (7.3) |
| Participants with any TEAE leading to study | 0 | | 1 | (2.4) |
| drug discontinuation^{†} | | | | |
| Participants with any TESAE^{‡} | 5 | (26.3) | 7 | (17.1) |
| Participants with any TEAE leading to study | 0 | | 0 | |
| withdrawal | | | | |
| Participants with any TEAE leading to death | 0 | | 0 | |
| Participants with any TEAESI | | | | |

| | | | |
|---|---|---|---|
| ALT or AST elevations >3 × ULN | 0 | 10 | (24.4) |
| Suspected or confirmed thromboembolic | 0 | 2 | (4.9) |
| events | | | |

| | | |
|---|---|---|
| Severe or serious injection site reactions | 0 | 0 |
| Systemic injection-associated reactions | 0 | 0 |

*Three participants in China were treated but not randomised; participants were enrolled into a China-specific non-randomised fitusiran group to receive subcutaneous fitusiran prophylaxis every 4 weeks, with initial starting dose of 50 mg.
^{†}In the fitusiran 80 mg prophylaxis group, one (2.4%) participant experienced two TEAEs that resulted in study drug discontinuation (spinal vascular disorder and thrombosis [suspected spinal vessel thrombosis]).
^{‡}TESAEs were reported in seven participants in the fitusiran prophylaxis group (asymptomatic COVID-19, device-related infection, haematoma infection, vascular device infection, anaemia, spinal vascular disorder, subclavian vein thrombosis, thrombosis, biliary colic, acute cholecystitis, chronic cholecystitis, and haematuria [all n = 1]). In the BPA on-demand group, TESAEs were reported in five participants (haemorrhage, haemarthrosis, muscle haemorrhage, haematuria, tooth fracture, and traumatic haemorrhage [all n = 1]).
Abbreviations are as follows: ALT, alanine transaminase; AST, aspartate aminotransferase; BPA, bypassing agent; TEAE, treatment-emergent adverse event; TESAE, treatment-emergent serious adverse event; TEAESI, adverse event of special interest; ULN, upper limit of normal.

In the fitusiran prophylaxis group, TEAESIs of “any ALT or AST elevations >3×ULN” were reported in ten (24%) participants (Table 20). For most participants, event onset occurred within 60 days of fitusiran initiation. All events were classified by the investigator as non-serious and mild-to-moderate in severity. These events resulted in interruption of fitusiran prophylaxis in three (7%) participants, which was required due to ALT or AST elevations >5×ULN, and in discontinuation of fitusiran prophylaxis in no participants. In all three participants with an interruption in fitusiran prophylaxis, the events were reported by the Investigator as recovered or resolved; median time to event resolution was 42 days (minimum five days; maximum 50 days). Recurrent elevations >3×ULN were reported in one of these participants. Of the seven participants who continued on fitusiran prophylaxis, the events were reported by the Investigator as recovered or resolved in four participants; median time to event resolution was 76.5 days (minimum 24 days; maximum 148 days). Recurrent elevations >3×ULN were reported in one of these participants. Laboratory abnormalities consistent with Hy's Law were not identified. There were no reports of “any ALT or AST elevations >3×ULN” in the BPA on-demand group.

In the fitusiran prophylaxis group, four TEAESIs of ‘suspected or confirmed thromboembolic events’ were reported in two (5%) participants (Table 20). These events were deep vein thrombosis (non-serious), subclavian vein thrombosis (serious), and superficial thrombophlebitis (non-serious) involving the upper extremities in a single participant in the setting of central venous access and associated complications. All three of these events resulted in interruption of fitusiran and were assessed by the investigator as unlikely related to the study drug. The antithrombin value in this participant prior to onset of these events was 11.9%. In a second participant, an event of thrombosis (suspected spinal vessel thrombosis) was assessed by the investigator as serious and possibly related to the study drug, resulting in discontinuation of fitusiran prophylaxis. Steady state antithrombin values in this participant prior to event onset ranged from 7.8% to 11.6%. There were no TEAESIs of “any suspected or confirmed thromboembolic events” reported in the BPA on-demand group.

There were no TEAESIs of severe or serious injection site reactions or systemic injection associated reactions reported.

In the fitusiran group, six participants experienced TEAEs of cholecystitis and/or cholelithiasis. Of those six participants, cholecystitis and cholelithiasis were reported concomitantly in three participants, acalculous cholecystitis was reported in one participant, and cholelithiasis alone was reported in two participants. Events of cholecystitis and/or cholelithiasis were managed via cholecystectomy in one participant. There were no events of cholecystitis or cholelithiasis reported in the BPA on-demand group.

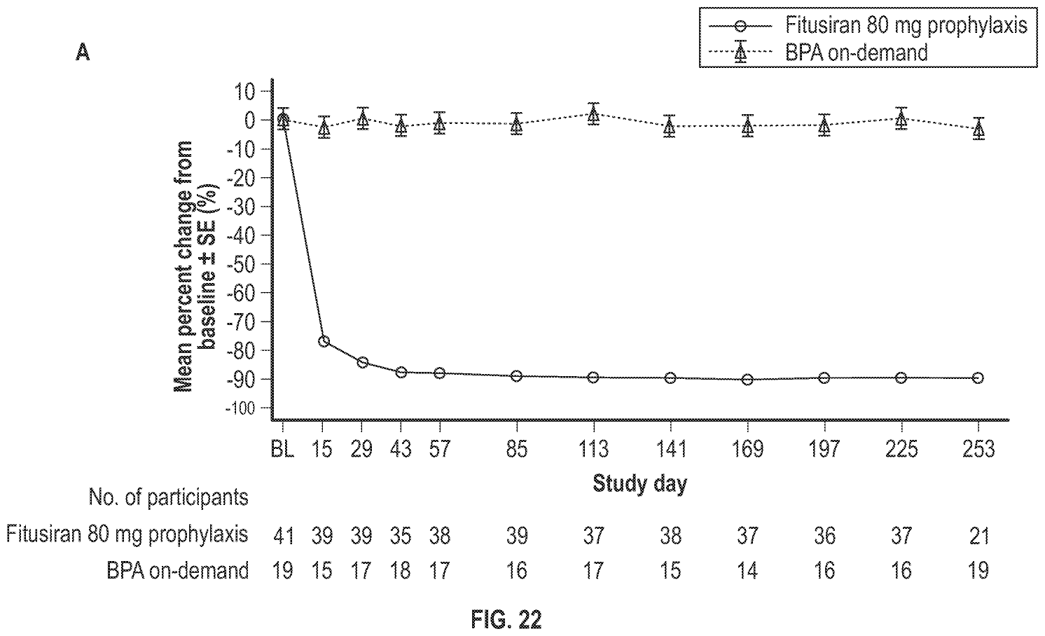

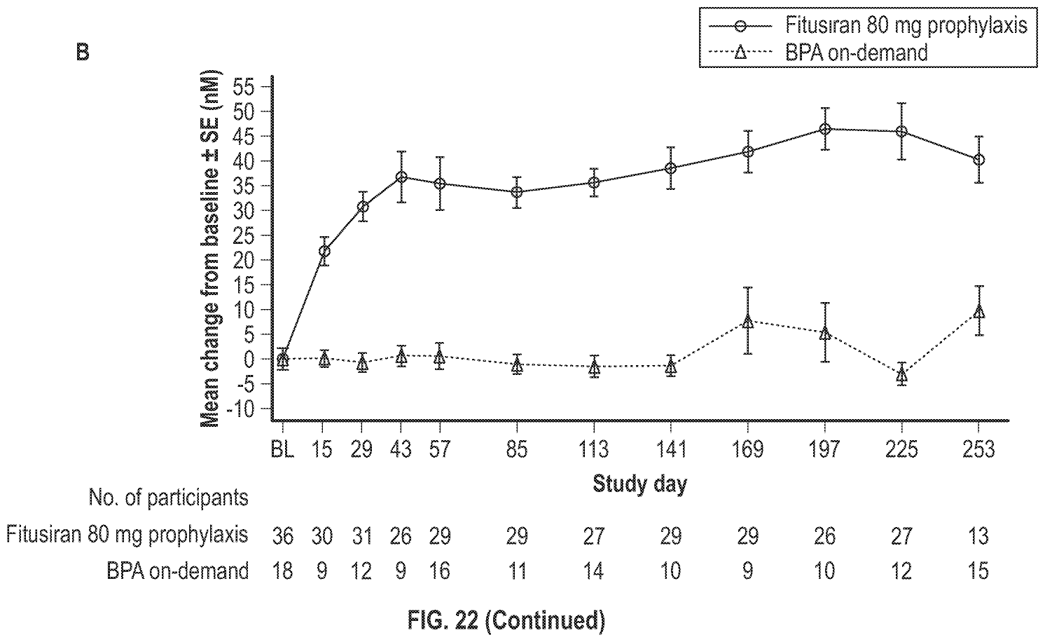

Once-monthly dosing with fitusiran prophylaxis resulted in a sustained lowering of antithrombin activity levels from mean 104% (SD: 10.5) at baseline, with a 77% mean reduction (SD: 8.6) from baseline by day 15, and an 84% mean reduction (SD: 7.2) by day 29 (FIG. 22 panel A). Antithrombin levels remained reduced by 84 to 90% from day 29 to trial end. At study endpoint, mean AT levels were 11% (SD: 2.9). As expected, treatment with BPA had no effect on antithrombin levels. There was a mean (SD) increase in peak thrombin generation of 21.9 nM (16.4) from baseline in the fitusiran group on Day 15, increasing to a peak mean thrombin generation of 40.3 nM (17.9) on Day 29 and 46.7 nM (23.0) by Day 43 (corresponding to a 540% change from baseline) that remained elevated during the trial in the fitusiran prophylaxis group (FIG. 22 panel B).

Overall, one (3%) participant in the fitusiran prophylaxis group was confirmed ADA positive at Day 29, at a low ADA titer of 50 (equivalent to minimum required dilution of the assay); this was transient, and the participant was negative at subsequent assessments in month 3 and 8.

### Conclusion

In people with hemophilia A or B with inhibitors, once-monthly, 80 mg subcutaneous, prophylactic fitusiran resulted in a median ABR of zero, a high rate (65.8%) of participants with zero bleeds, and improvements in quality of life, thereby meeting the primary endpoint of the study. These results demonstrate the efficacy of fitusiran prophylaxis in providing consistent protection against bleeding in those with inhibitors.

1. A method of improving patient-reported outcome (PRO) in a hemophilia patient, comprising subcutaneously administering a therapeutically effective amount of fitusiran to the hemophilia patient in need thereof, optionally wherein the patient is a hemophilia A or B patient with or without inhibitors and optionally wherein the PRO is improved in one or more quality of life (QoL) domains.

2. A method of improving quality of life (QoL) in a hemophilia patient, comprising subcutaneously administering fitusiran to the hemophilia patient in need thereof, wherein the QoL is improved in one or more QOL domains and optionally wherein the patient is a hemophilia A or B patient with or without inhibitors.

3. The method of claim 1 or 2, wherein fitusiran is administered at about 10 to about 90 mg per dose.

4. The method of any one of claims 1-3, wherein the one or more QoL domains are domains in a QoL questionnaire, optionally wherein the QoL questionnaire is Haemophilia Quality of Life Questionnaire for Adults (Haem-A-QoL).

5. The method of claim 4, wherein the administering results in a clinically meaningful improvement indicated by a reduction of 7 or more units (optionally 8 or more, 9 or more, or 10 or more units) in one or more of Total Score, Sports and Leisure domain score, and Physical Health domain score of the questionnaire.

6. The method of any one of claims 1-3, wherein the one or more QoL domains are domains in a QoL questionnaire, optionally wherein the QoL questionnaire is Haemophilia Quality of Life Questionnaire for children and adolescents (Haemo-QoL).

7. The method of claim 6, wherein the administering results in a clinically meaningful improvement indicated by a reduction of 7 or more units (optionally 8 or more, 9 or more, or 10 or more units) in the Total Score of the questionnaire.

8. The method of any one of claims 1-3, wherein the one or more QoL domains are domains in a QoL questionnaire, optionally wherein the QoL questionnaire is Hemophilia Activities List (HAL).

9. The method of claim 8, wherein the administering results in an improvement indicated by an increase of 7 or more units (optionally 8 or more, 9 or more, or 10 or more units) in the Total Score of the questionnaire.

10. The method of any one of claims 1-3, wherein the one or more QoL domains are domains in a QoL questionnaire, optionally wherein the QoL questionnaire is pediatric Hemophilia Activities List (pedHAL).

11. The method of claim 10, wherein the administering results in an improvement indicated by an increase of 7 or more units (optionally 8 or more, 9 or more, or 10 or more units) in the Total Score of the questionnaire.

12. The method of any one of claims 1-3, wherein the one or more QoL domains are domains in a QoL questionnaire, optionally wherein the QoL questionnaire is Treatment Satisfaction Questionnaire for Medication Version 9 (TSQM-9).

13. The method of claim 12, wherein the administering results in an improvement indicated by an increase of 7 or more units (optionally 8 or more, 9 or more, or 10 or more units) in one or more of Effectiveness, Satisfaction, and Convenience domain score of the questionnaire.

14. The method of any one of claims 1-3, wherein the one or more QoL domains are domains in a QoL questionnaire, optionally wherein the QoL questionnaire is EuroQol 5-Dimensions (EQ-5D-5L).

15. The method of claim 14, wherein the administering results in an improvement indicated by an increase of 0.02 or more units (optionally 0.03 or more, 0.04 or more, or 0.05 or more units) in the total score of the questionnaire or an increase of 5 or more units (optionally 6 or more, 7 or more, or 8 or more units) in the visual analog scale (VAS) score of the questionnaire.

16. The method of any one of claims 1-15, wherein the patient is an adult or adolescent patient twelve years or older with hemophilia A or B with or without inhibitors.

17. The method of any one of claims 1-16, wherein the patient has hemophilia A.

18. The method of claim 17, wherein the patient has been treated with factor VIII or a bypassing agent (BPA).

19. The method of claim 18, wherein the patient is with inhibitors, optionally wherein the level of inhibitors is more than 0.6 BU/mL as determined by Bethesda inhibitor assay.

20. The method of claim 17 or 18, wherein the patient is without inhibitors.

21. The method of any one of claims 1-16, wherein the patient has hemophilia B.

22. The method of claim 21, wherein the patient has been treated with factor IX or a BPA.

23. The method of claim 22, wherein the patient is with inhibitors, optionally wherein the level of inhibitors is more than 0.6 BU/mL as determined by Bethesda inhibitor assay.

24. The method of claim 21 or 22, wherein the patient is without inhibitors.

25. The method of any one of claim 18-20 or 22-24, wherein the BPA is activated prothrombin complex concentrates (aPCC) and/or recombinant activated Factor VII (rFVIIa).

26. The method of any one of claims 1-25, comprising administering to the patient a plurality of doses of fitusiran at about 10 mg, about 20 mg, about 50 mg, or about 80 mg per dose.

27. The method of any one of claims 1-26, wherein fitusiran is administered to the patient about once every four weeks or about once a month or about once every eight weeks or about once every two months.

28. The method of any one of claims 1-27, wherein fitusiran is provided at a concentration of about 1 to about 200 mg/mL, optionally at a concentration of about 6.25, about 12.5 mg/mL, or about 100 mg/mL, in a phosphate-buffered saline (pH 7).

29. The method of any one of claims 1-28, wherein the patient does not have
(i) presence of clinically significant liver disease,
(ii) ALT >1.5×upper limit of normal reference range (ULN),
(iii) AST >1.5×upper limit of normal reference range (ULN),
(iv) hepatitis C,
(v) hepatitis A,
(vi) hepatitis E, and/or
(vii) hepatitis B.

30. Fitusiran for use in the method of any one of claims 1-29.

31. An article of manufacture for use in the method of any one of claims 1-29.

32. The article of manufacture for use of claim 31, wherein the article of manufacture is a single-use container containing
i. about 80 mg of fitusiran in about 0.8 mL of a phosphate-buffered saline (pH 7),
ii. about 50 mg of fitusiran in about 0.5 mL of a phosphate-buffered saline (pH 7),
iii. about 20 mg of fitusiran in about 0.2 mL of a phosphate-buffered saline (pH 7), or
iv. about 10 mg of fitusiran in about 0.1 mL of a phosphate-buffered saline (pH 7).

33. The article of manufacture for use of claim 31, wherein the article of manufacture is a single-use prefilled syringe containing
i. about 80 mg of fitusiran in about 0.8 mL of a phosphate-buffered saline (pH 7),
ii. about 50 mg of fitusiran in about 0.5 mL of a phosphate-buffered saline (pH 7),
iii. about 20 mg of fitusiran in about 0.2 mL of a phosphate-buffered saline (pH 7), or
iv. about 10 mg of fitusiran in about 0.1 mL of a phosphate-buffered saline (pH 7).

34. Use of fitusiran for the manufacture of a medicament to treat hemophilia A or B in the method of any one of claims 1-29.
